(19) United States
(12) Patent Application Publication
(10) Pub. No.: US 2025/0163139 A1
(43) Pub. Date: May 22, 2025

(54) ANTIBODY THAT BINDS TO VEGF-A AND IL6 AND METHODS OF USE

(71) Applicant: Hoffmann-La Roche Inc., Little Falls, NJ (US)

(72) Inventors: Roland BECKMANN, Vienna (AT); Joerg BENZ, Rheinfelden (DE); Davide BERTOLDO, München (DE); Faye Marie DRAWNEL, Binningen (CH); Derrick FEENSTRA, Basel (CH); Sebastian FENN, Achmühle/Eurasburg (DE); Stefan FROST, Bad Heilbrunn (DE); Marlon HINNER, München (DE); Kristian Hobolt JENSEN, Copenhagen (DK); Daniela MATSCHEKO, Kochel (DE); Anastasia MEIER, München (DE); Oliver PLOETTNER, Gilching (DE); Janina SPECK, Penzberg (DE); Pamela STRASSBURGER, Weil am Rhein (DE); Barbara Marie-Luise WEISER, Grenzach-Wyhl (DE); Gabriella WIDMER, Reinach (CH)

(73) Assignee: Hoffmann-La Roche Inc., Little Falls, NJ (US)

(21) Appl. No.: 18/931,542

(22) Filed: Oct. 30, 2024

Related U.S. Application Data
(63) Continuation of application No. PCT/EP2023/062545, filed on May 11, 2023.

(30) Foreign Application Priority Data
May 11, 2022 (EP) 22172759.7

(51) Int. Cl. C07K 16/22; C07K 16/24

(52) U.S. Cl. CPC C07K 16/22; C07K 16/248; C07K 2317/14; C07K 2317/31; C07K 2317/55; C07K 2317/565; C07K 2317/76; C07K 2317/92

(57) ABSTRACT

The present invention relates to anti-VEGF-A/anti-IL6 antibodies. e.g. in the form of a bispecific Fab fragment, and methods of using the same.

### CROSS-REFERENCE TO RELATED APPLICATIONS

This application is a Continuation of International Application No. PCT/EP2023/062545, filed May 11, 2023, which claims benefit of priority to European Application No. 22172759.7 filed May 11, 2022, each of which is incorporated herein by reference in its entirety.

### SEQUENCE LISTING

This application contains a Sequence Listing which has been submitted electronically in XML format and is hereby incorporated by reference in its entirety. Said XML copy, created on Oct. 11, 2024, is named P37534-US_Sequence Listing.xml and is 63,986 bytes in size.

### FIELD OF THE INVENTION

The present invention relates to anti-VEGF-A/anti-IL6 antibodies and methods of using the same.

### BACKGROUND OF THE INVENTION

Antibodies binding to VEGF, e.g. ranibizumab, are used as therapeutic for therapy of ocular vascular diseases like age related macular degeneration. Antibodies binding to IL6, e.g. as disclosed in WO2014/074905, have been suggested for therapy of ocular diseases.

WO2012/163520 discloses bispecific antibodies comprising two paratopes in one pair of VH and VL domains (“DutaFabs”). Each paratope of the bispecific antibody of WO2012/163520 comprises amino acids from the heavy chain and from the light chain CDRs, wherein heavy chain CDR-H1 and CDR-H3 as well as light chain CDR-L2 contribute to the first paratope and light chain CDR-LI and CDR-L3 as well as heavy chain CDR-H2 contribute to the second paratope Monospecific antibodies comprising the individual paratopes are isolated independently from different Fab-libraries, which are diversified in either the first or the second paratope. The amino acid sequences of said monospecific antibodies are identified and merged into the biparatopic VH and VL pair. One exemplary Fab fragment, termed “VH6L” having a VL sequence of SEQ ID NO:01 and a VH sequence of SEQ ID NO:02, specifically binding to VEGF and IL-6 is disclosed in WO2012/163520 as a proof of concept example.

Indeed there is a need for improved therapeutic antibodies that bind to VEGF and and to IL6 for clinical application in ocular diseases, e.g. by improving efficacy vs. standard-of-care and by improving duration of action and in turn, reducing frequency of intravitreal injections, leading to less administration burden for the patient.

### SUMMARY OF THE INVENTION

The present invention relates to bispecific anti-VEGF-A/anti-IL6 antibodies and methods of using the same.

In one aspect the invention relates to an antibody that binds to human VEGF-A and to human IL6 comprising a VH domain comprising (a) CDR-H1 comprising the amino acid sequence of SEQ ID NO:18, (b) CDR-H2 comprising the amino acid sequence of SEQ ID NO: 19, and (c) CDR-H3 comprising the amino acid sequence of SEQ ID NO:20, and a VL domain comprising (d) CDR-LI comprising the amino acid sequence of SEQ ID NO: 15, (e) CDR-L2 comprising the amino acid sequence of SEQ ID NO: 16, and (f) CDR-L3 comprising the amino acid sequence of SEQ ID NO: 17, comprising a variable heavy chain domain comprising an amino acid sequence of SEQ ID NO:22 with up to 5 amino acid substitutions; and a variable light chain domain comprising an amino acid sequence of SEQ ID NO:21 with up to 5 amino acid substitutions.

One embodiment of the invention relates to an antibody that binds to human VEGF-A and to human IL6, comprising a VH sequence of SEQ ID NO:22 and a VL sequence of SEQ ID NO:21.

One embodiment of the invention relates to an antibody comprising a heavy chain amino acid sequence of SEQ ID NO:24 and a light chain amino acid sequence of SEQ ID NO:23.

One embodiment of the invention relates to an antibody Fab fragment that binds to human VEGF-A and to human IL6.

One embodiment of the invention relates to a bispecific antibody Fab fragment that binds to human VEGF-A and to human IL6.

In another aspect the invention provides an antibody that binds to IL6 that binds to the same epitope on IL6 as an antibody according to the invention.

In another aspect the invention provides an antibody that binds to human IL6 comprising;
a) a VH domain based on a human VH3 framework, wherein the IL6 paratope comprises amino acid residues Y1, I2, Q3, Y26, E27, F28, T29, H30, Q31, D32, P52a, R94, 196, D97, F98, D101, T102, and a VL domain based on a human Vkappal framework, wherein the IL6 paratope comprises amino acid residues Y49, D50, S53, N54, Y55, P56, S57, Y91, Y96; or
b) a VH domain based on a human VH3 framework, wherein the IL6 paratope comprises amino acid residues Y1, P2, Q3, V26, L27, F28, K29, H30, Q31, D32, P52a, R94, L96, D97, F98, D101, E102, and a VL domain based on a human Vkappal framework, wherein the IL6 paratope comprises amino acid residues Y49, D50, D53, R54, Y55, P56, E57, Y91, Y96 (numbering according to Kabat).

In another aspect the invention provides an antibody that binds to IL6 that binds to the same epitope on IL6 as an antibody with a VL domain of SEQ ID NO: 35 and a VH domain of SEQ ID NO: 36. In one embodiment the antibody comprises a VH domain having a human VH3 framework, wherein the IL6 paratope comprises amino acid residues 1, 2, 3, 26, 27, 28, 29, 30, 31, 32, 52a, 94, 96, 97, 98, 101, 102 and a VL domain having a human Vkappal framework, wherein the IL6 paratope comprises amino acid residues 49, 50, 53, 54, 55, 56, 57, 91, 96.

In one aspect, the invention provides an isolated nucleic acid encoding the antibody of the invention.

In one aspect, the invention provides a host cell comprising the nucleic acid of the invention. In one embodiment, the host cell is a CHO cell. In one embodiment, the host cell is an E. coli cell.

In one aspect, the invention provides an expression vector comprising the nucleic acid of the invention.

In one aspect, the invention provides a method of producing an antibody that binds to human VEGF-A and to human IL6 comprising culturing the host cell of the invention so that the antibody is produced.

In one aspect, the invention provides the antibody produced by the method of the invention.

In one aspect, the invention provides a pharmaceutical formulation comprising the antibody of the invention and a pharmaceutically acceptable carrier.

In one aspect, the invention provides a pre-filled syringe comprising the antibody of the invention and a pharmaceutically acceptable carrier.

In one aspect, the invention provides an ocular implant comprising the antibody of the invention and a pharmaceutically acceptable carrier. In one embodiment, the invention comprises a port delivery device comprising the antibody of the invention.

In one aspect of the invention, a port delivery device administers the antibody or the pharmaceutical formulation.

In one aspect, the invention provides the antibody of the invention for use as a medicament, in one embodiment for use in the treatment of a vascular disease.

In one aspect, the invention provides the use of the antibody of the invention or the pharmaceutical composition of the invention in the manufacture of a medicament, in one embodiment a medicament for the treatment of a vascular disease.

In one aspect, the invention provides a method of treating an individual having a vascular disease comprising administering to the individual an effective amount of the antibody of the invention or the pharmaceutical composition of the invention.

In one aspect, the invention provides a method of inhibiting angiogenesis in an individual comprising administering to the individual an effective amount of the antibody of the invention or the pharmaceutical composition of the invention to inhibit angiogenesis.

According to the invention a therapeutic anti-VEGF-A/anti-IL6 antibody is provided that is capable of binding to its target antigens independently, even when it is provided in the form of an antibody Fab fragment. It exhibits a superior KD and species cross-reactivity with cynomolgus targets within a pharmacologically relevant range. The antibody of the invention is suitable for the treatment of ocular vascular diseases. The antibody of the invention provides several valuable properties, including good expressibility and developability (e.g. high binding potency, high biophysical and biochemical stability, high concentration formulation) that allow its therapeutic application, particularly high affinities to both targets supporting a low efficacious dose, and a high stability advantageous for long duration. Compared to non-antibody approaches the antibody of the invention tends to be more acceptable due to its high humanness and lack of artificial domains and linkers. In addition, the antibody of the invention is advantageous to be provided in high-concentration liquid formulations with a viscosity suitable for ocular application. Because it can be provided in high concentrations, treatment with an antibody of the invention is more acceptable for a patient as a higher dose of the therapeutic can be applied at one treatment allowing for a longer treatment cycle. A bispecific Fab fragment such as the one described in this invention has an additional advantage over a bispecific full-length IgG antibody due to the much lower molecular weight. While the Fab has a molecular weight of approx. 50 kDa, the full length antibody's weight is three times as high (approx. 150 kDa) while providing the same number of binding sites. For a given amount of drug, a bispecific Fab fragment will therefore contain three times more binding sites compared to the full length IgG antibody.

### DESCRIPTION OF THE FIGURES

FIG. 1: Binding of parental bispecific antibodies 6HVL_1 and V6HL_1 to human and cynomolgus IL6 as determined by Surface plasmon resonance

FIG. 2: VEGF IC50 of parental bispecific antibodies 6HVL_1 and V6HL_1 using human VEGF-165

FIG. 3: Binding of improved bispecific antibodies to human and cynomolgus IL6 as determined by Surface plasmon resonance.

FIG. 4: VEGF IC50 of improved bispecific antibodies using human VEGF-121 and VEGF-165.

FIG. 5: Crystal structure of Fab0182-IL-6 complex. Overall view onto the structure of IL-6 bound to Fab 0182. IL-6 is colored in salmon, the light and heavy chain of Fab 0182 are colored in cyan and blue, respectively.

FIG. 6: Crystal structure of Fab 6HVL4.1-IL-6 complex. Overall view onto the structure of IL-6 bound to Fab 6HVL4.1. IL-6 is colored in salmon, the light and heavy chain of Fab 6HVL4.1 are colored in wheat and blue, respectively.

FIG. 7: Simultaneous binding of anti-VEGF/anti-IL-6 Fab to its targets as assessed by SPR with immobilized anti-Fab antibody

FIG. 8: Blocking of VEGF-R2 binding by anti-VEGF/anti-IL-6 Fab in presence of IL-6 as assessed by SPR with immobilized VEGF-A

FIG. 9: Influence of VEGF-binding on IL6 activity was assessed as follows by a cell-based IL-6 specific reporter gene assay (without preincubation)

FIG. 10: Influence of VEGF-binding on IL6 activity was assessed as follows by a cell-based IL-6 specific reporter gene assay (with preincubation)

FIG. 11: Inhibition of IL-6 Signaling in HRMECs by 6HVL_4

FIG. 12: Dose-Dependent Change in the % Inhibition of VEGF-A Induced HUVEC Proliferation by 6HVL_4

FIG. 13: Recovery of IL6/IL6R/VEGF induced barrier breakdown on HRMVECs by 6HVL_4

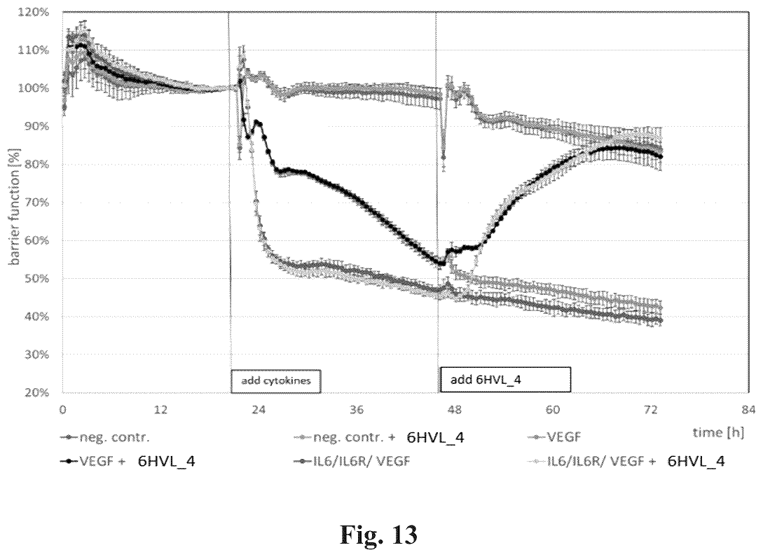

FIG. 14: Recovery of IL6/IL6R/VEGF induced barrier breakdown on HRMVECs by aflibercept

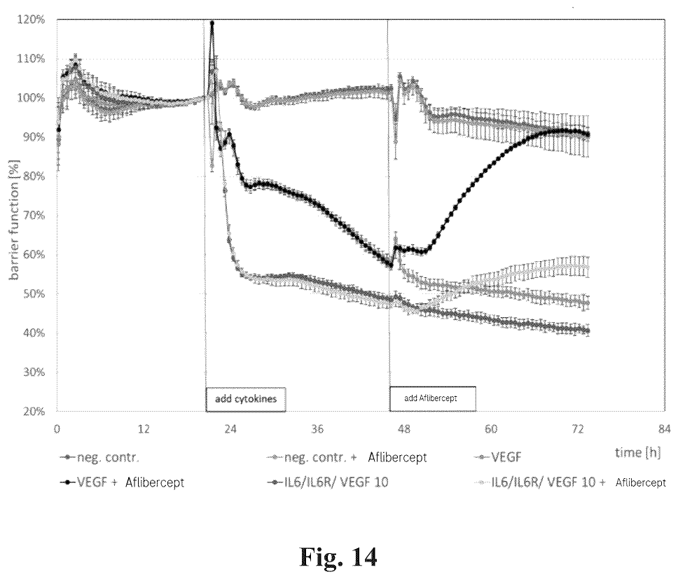

FIG. 15: Amino acid sequences of VH and VL domains of indicated antibodies. Kabat numbering of the amino acid position is indicated. Amino acid positions contributing to IL6 paratope as identified in Example 13 are highlighted by the black boxes.

FIG. 16: Image of IL6/IL6R/gp130 complex (top: pdb-acc. #1p9m) in comparison to an overlay of structures of Fab 0182 bound to IL6 and of the IL6/IL6R complex from pdb-acc. 1p9m (bottom).

FIG. 17: Image of IL6/IL6R/gp130 complex (top: pdb-acc. #1p9m) in comparison to an overlay of structures of Fab 6HVL4.1 bound to IL6 and of the IL6/IL6R complex from pdb-acc. 1p9m (bottom).

FIG. 18: SPR bridging experiment investigating the ability IL6R to bind to IL6 when engaged in a preformed complex with Fab PIAE2421.

FIG. 19: SPR binding experiment investigating the ability of Fab PIAE2421 to bind human “hyper-IL6” (a chimera of human IL6 and IL6R).

FIG. 20: ELISA competition experiment determining the ability of Fab PIAE2421 to bind to IL6 in a manner that blocks the binding of IL6 to IL6R.

FIG. 21: Binding of IL6 binding antibody 6HdL2.05 to human and cynomolgus IL6 as determined by Surface plasmon resonance

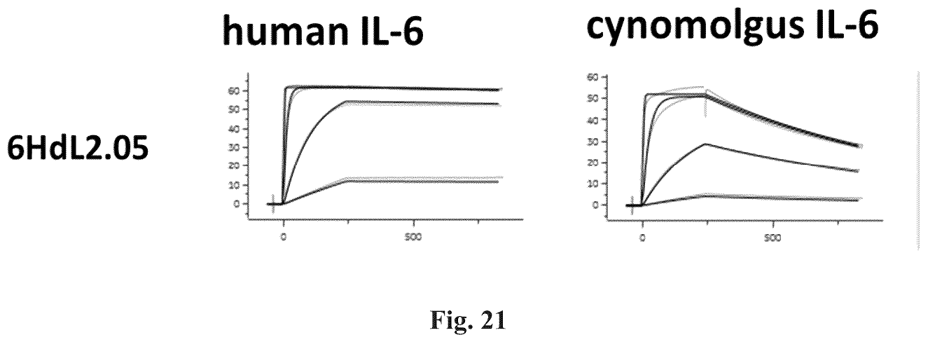

### DETAILED DESCRIPTION OF THE INVENTION

### 1. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

Unless otherwise defined herein the term “comprising of” shall include the term “consisting of”.

The term “about” as used herein in connection with a specific value (e.g. temperature, concentration, time and others) shall refer to a variation of +/−1% of the specific value that the term “about” refers to.

The term “antibody” herein is used in the broadest sense and encompasses various antibody structures, including but not limited to monoclonal antibodies, multispecific antibodies (e.g., bispecific antibodies), and antibody fragments so long as they exhibit the desired antigen-binding activity.

An “isolated” antibody is one, which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method.

The terms “full length antibody”, “intact antibody”, and “whole antibody” are used herein interchangeably to refer to an antibody having a structure substantially similar to a native antibody structure or having heavy chains that contain an Fc region as defined herein.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain embodiments, the antibody is of the IgG1 isotype. In certain embodiments, the antibody is of the IgG1 isotype with the P329G, L234A and L235A mutation to reduce Fc-region effector function. In other embodiments, the antibody is of the IgG2 isotype. In certain embodiments, the antibody is of the IgG4 isotype with the S228P mutation in the hinge region to improve stability of IgG4 antibody. The heavy chain constant domains that correspond to the different classes of immunoglobulins are called α, δ, ∈, γ, and μ, respectively. The light chain of an antibody may be assigned to one of two types, called kappa (κ) and lambda (λ), based on the amino acid sequence of its constant domain.

The term “Fc region” herein is used to define a C-terminal region of an immunoglobulin heavy chain that contains at least a portion of the constant region.

The term includes native sequence Fc regions and variant Fc regions. In one embodiment, a human IgG heavy chain Fc region extends from Cys226, or from Pro230, to the carboxyl-terminus of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) (see, e.g., Kindt et al. Kuby Immunology, 6th ed., W. H. Freeman and Co., page 91 (2007)). In the antibody of the invention, a single pair of a VH domain and a VL domain, i.e. a cognate VH/VL pair, specifically binds to its two targets: VEGF-A and IL6.

A “DutaFab” is a bispecific antibody as disclosed in WO2012/163520. In a DutaFab a single pair of a VH domain and a VL domain specifically binds to two different epitopes, wherein one paratope comprises amino acid residues from CDR-H2, CDR-L1 and CDR-L3 and the other paratope comprises amino residues from CDR-H1, CDR-H3 and CDR-L2. DutaFabs comprise two non-overlapping paratopes within a cognate VH/VL pair and may simultaneously bind to the two different epitopes. DutaFabs and methods for their generation by screening of libraries comprising monospecific Fab fragments are disclosed in WO2012/163520.

A “human antibody” is one, which possesses an amino acid sequence, which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Antibodies or antibody fragments isolated from human antibody libraries are considered human antibodies or human antibody fragments herein.

A “human consensus framework” is a framework, which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2: diabodies; linear antibodies; single-chain antibody molecules (e.g. scFv); and multispecific antibodies formed from antibody fragments.

A “paratope” or “antigen binding site”, as used interchangeably herein, refers to a part of an antibody which recognizes and binds to an antigen. A paratope is formed by several individual amino acid residues from the antibody's heavy and light chain variable domains arranged that are arranged in spatial proximity in the tertiary structure of the Fv region. The antibodies of the invention comprise two paratopes in one cognate VH/VL pair.

As used herein a “VEGF-A paratope” is a paratope or antigen binding site that binds to VEGF-A. The VEGF-A paratope of an antibody of the invention comprises amino acid residues from CDR-H2, CDR-LI and CDR-L3 of the antibody.

As used herein an “IL6 paratope” is a paratope or antigen binding site that binds to IL6. The IL6 paratope of an antibody of the invention comprises amino acid residues from CDR-H1, CDR-H3 and CDR-L2 of the antibody.

The term “vascular endothelial growth factor”, abbreviated “VEGF”, as used herein, refers to any native VEGF from any vertebrate source, including mammals such as primates (e.g. humans) and rodents (e.g., mice and rats), unless otherwise indicated. The term encompasses “full-length”, unprocessed VEGF as well as any form of VEGF that results from processing in the cell. The term also encompasses naturally occurring variants of VEGF, e.g., splice variants or allelic variants. The amino acid sequence of an exemplary human VEGF is shown in SEQ ID NO:27.

The terms “anti-VEGF-A antibody” and “an antibody that binds to VEGF-A” refer to an antibody that is capable of binding VEGF-A with sufficient affinity such that the antibody is useful as a diagnostic and/or therapeutic agent in targeting VEGF-A.

In one embodiment, the extent of binding of an anti-VEGF-A antibody to an unrelated, non-VEGF-A protein is less than about 10% of the binding of the antibody to VEGF-A as measured, e.g., by surface plasmon resonance (SPR). In certain embodiments, an antibody that binds to VEGF-A has a dissociation constant (KD) of ≤1 nM, ≤0.1 nM, or ≤0.01 nM. An antibody is said to “specifically bind” to VEGF-A when the antibody has a K_{D} of 1 μM or less.

The term “Interleukin-6”, abbreviated “IL6”, as used herein, refers to any native IL6 from any vertebrate source, including mammals such as primates (e.g. humans) and rodents (e.g., mice and rats), unless otherwise indicated. The term encompasses “full-length”, unprocessed IL6 as well as any form of IL6 that results from processing in the cell. The term also encompasses naturally occurring variants of IL6, e.g., splice variants or allelic variants. The amino acid sequence of an exemplary human IL6 is shown in SEQ ID NO:28.

An antibody of the invention “simultaneously binds to human VEGF-A and human IL6”, which means that (a) an antibody Fab fragment of the invention that is bound to human IL6 (also) specifically binds to human VEGF-A, and (b) an antibody Fab fragment of the invention that is bound to human VEGF-A (also) specifically binds to human IL6. Simultaneous binding may be assessed with methods known in the art, e.g. by surface plasmon resonance as described herein.

The term “complementarity determining regions” or “CDRs” as used herein refers to each of the regions of an antibody variable domain, which are hypervariable in sequence and contain antigen-contacting residues. Generally, antibodies comprise six CDRs: three in the VH domain (CDR-H1, CDR-H2, CDR-H3), and three in the VL domain (CDR-L1, CDR-L2, CDR-L3). Unless otherwise indicated, CDR residues and other residues in the variable domain (e.g., FR residues) are numbered herein according to the Kabat numbering system (Kabat et al., Sequences of Proteins of Immunological Interest, 5th Ed. Public Health Service, National Institutes of Health, Bethesda, MD, 1991).

“Framework” or “FR” as used herein refers to variable domain amino acid residues other than CDR residues. The framework of a variable domain generally consists of four framework domains: FRI, FR2, FR3, and FR4. Accordingly, the CDR and FR amino acid sequences generally appear in the following sequence in the (a) VH domain: FR1-CDR-H1-FR2-CDR-H2-FR3-CDR-H3-FR4; and (b) in the VL domain: FRI-CDR-LI-FR2-CDR-L2-FR3-CDR-L3-FR4.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary embodiments for measuring binding affinity are described herein.

The term “epitope” denotes the site on an antigen, either proteinaceous or non-proteinaceous, to which an antibody binds. Epitopes can be formed both from contiguous amino acid stretches (linear epitope) or comprise non-contiguous amino acids (conformational epitope), e.g. coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to VEGF-A or IL6 based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

In addition, competitive binding can be used to easily determine whether an antibody binds to the same epitope of VEGF-A or IL6 as, or competes for binding with, a reference antibody of the invention. For example, an “antibody that binds to the same epitopes on VEGF-A and IL6” as a reference-antibody refers to an antibody that blocks binding of the reference-antibody to its antigens in respective competition assay's by 50% or more, and conversely, the reference antibody blocks binding of the antibody to its antigen in respective competition assays by 50% or more. Also for example, to determine if an antibody binds to the same epitope as a reference-antibody, the reference-antibody is allowed to bind to VEGF-A or IL6 under saturating conditions. After removal of the excess of the reference-antibody, the ability of an antibody in question to bind to VEGF-A or IL6 is assessed. If the antibody in question is able to bind to VEGF-A or IL6 after saturation binding of the reference-antibody, it can be concluded that the antibody in question binds to a different epitope than the reference-antibody. But, if the antibody in question is not able to bind to VEGF-A or IL6 after saturation binding of the reference-antibody, then the antibody in question may bind to the same epitope as the epitope bound by the reference-antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to VEGF-A or IL6, then it can be concluded that the antibody in question and the reference-antibody compete for binding to VEGF-A or IL6.

In some embodiments two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some embodiments, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2000/005319.

Unless otherwise indicated, For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448: W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or WWW.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open;-10; ext;-2; Ktup=2) to ensure a global, rather than local, alignment is performed. Percent amino acid identity is given in the output alignment header.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g. complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g. in a host or patient. Such DNA (e.g. cDNA) or RNA (e.g. mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g. Stadler et al, Nature Medicine 2017, published online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding” an antibody refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

An “effective amount” of an agent, e.g., a pharmaceutical composition, refers to an amount effective, at dosages and for periods of time necessary, to achieve the desired therapeutic or prophylactic result.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “ocular disease,” as used herein, includes any ocular disease associated with pathological angiogenesis and/or atrophy. An ocular disease may be characterized by altered or unregulated proliferation and/or invasion of new blood vessels into the structures of ocular tissues such as the retina or cornea. An ocular disease may be characterized by atrophy of retinal tissue (photoreceptors and the underlying retinal pigment epithelium (RPE) and choriocapillaris). Non-limiting ocular diseases include, for example, AMD (e.g., wet AMD, dry AMD, intermediate AMD, advanced AMD, and geographic atrophy (GA)), macular degeneration, macular edema, DME (e.g., focal, non-center DME and diffuse, center-involved DME), retinopathy, diabetic retinopathy (DR) (e.g., proliferative DR (PDR), non-proliferative DR (NPDR), and high-altitude DR), other ischemia-related retinopathies, ROP, retinal vein occlusion (RVO) (e.g., central (CRVO) and branched (BRVO) forms), CNV (e.g., myopic CNV), corneal neovascularization, diseases associated with corneal neovascularization, retinal neovascularization, diseases associated with retinal/choroidal neovascularization, central serous retinopathy (CSR), pathologic myopia, von Hippel-Lindau disease, histoplasmosis of the eye, FEVR, Coats' disease, Norrie Disease, retinal abnormalities associated with osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, including but not limited to CMV retinitis, ocular melanoma, retinal blastoma, conjunctivitis (e.g., infectious conjunctivitis and non-infectious (e.g,. allergic) conjunctivitis), Leber congenital amaurosis (also known as Leber's congenital amaurosis or LCA), uveitis (including infectious and non-infectious uveitis), choroiditis (e.g., multifocal choroiditis), ocular histoplasmosis, blepharitis, dry eye, traumatic eye injury, Sjögren's disease, and other ophthalmic diseases wherein the disease or disease is associated with ocular neovascularization, vascular leakage, and/or retinal edema or retinal atrophy. Additional exemplary ocular diseases include retinoschisis (abnormal splitting of the retina neurosensory layers), diseases associated with rubeosis (neovascularization of the angle) and diseases caused by the abnormal proliferation of fibrovascular or fibrous tissue, including all forms of proliferative vitreoretinopathy. Exemplary diseases associated with corneal neovascularization include, but are not limited to, epidemic keratoconjunctivitis, vitamin A deficiency, contact lens overwear, atopic keratitis, superior limbic keratitis, terygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegener's sarcoidosis, scleritis, Stevens-Johnson syndrome, periphigoid radial keratotomy, and corneal graph rejection. Exemplary diseases associated with choroidal neovascularization and defects in the retina vasculature, including increased vascular leak, aneurisms and capillary drop-out include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, retina edema (including macular edema), Eales disease, Behcet's disease, infections causing retinitis or choroiditis (e.g., multifocal choroidits), presumed ocular histoplasmosis, Best's disease (vitelliform macular degeneration), myopia, optic pits, pars planitis, retinal detachment (e.g., chronic retinal detachment), hyperviscosity syndromes, toxoplasmosis, trauma, and post-laser complications. Exemplary diseases associated with atrophy of retinal tissues (photoreceptors and the underlying RPE) include, but are not limited to, atrophic or nonexudative AMD (e.g., geographic atrophy or advanced dry AMD), macular atrophy (e.g., atrophy associated with neovascularization and/or geographic atrophy), diabetic retinopathy, Stargardt's disease, Sorsby Fundus Dystrophy, retinoschisis and retinitis pigmentosa.

The term “package insert” is used to refer to instructions customarily included in commercial packages of therapeutic products, that contain information about the indications, usage, dosage, administration, combination therapy, contraindications and/or warnings concerning the use of such therapeutic products.

### 2. Detailed Description of the Embodiments of the Invention

In one aspect, the invention is based, in part, on the provision of bispecific antibodies for therapeutic application. In certain aspects, antibodies that bind to human VEGF-A and human IL6 are provided. Antibodies of the invention are useful, e.g., for the treatment of vascular diseases, e.g. ocular vascular diseases.

A. Exemplary Antibodies that Bind to Human VEGF-A and Human IL6

In one aspect, the invention provides antibodies that bind to human VEGF-A and human IL6. In one aspect, provided are isolated antibodies that bind to human VEGF-A and human IL6. In one aspect, the invention provides antibodies that specifically bind to human VEGF-A and human IL6.

In certain aspects, an antibody that binds to human VEGF-A and to human IL6 is provided, wherein the antibody comprises a VEGF-A paratope (i.e. an antigen binding site that binds to VEGF-A) and an IL6 paratope (i.e. an antigen binding site that binds to IL6) within one cognate pair of a VL domain and a VH domain, wherein,
the VEGF-A paratope comprises amino acid residues from CDR-H2, CDR-LI and CDR-L3 of the antibody, wherein the IL6 paratope comprises amino acid residues from the CDR-H1, CDR-H3 and CDR-L2 of the antibody; and/or
the IL6 paratope comprises amino acid residues from CDR-H2, CDR-L1 and CDR-L3 of the antibody, wherein the VEGF-A paratope comprises amino acid residues from the CDR-H1, CDR-H3 and CDR-L2 of the antibody;
the pair of the variable light chain domain and the variable heavy chain domain simultaneously binds to human VEGF-A and human IL6; and/or
the antibody binds to the same epitope on human VEGF-A and to the same epitope on human IL6 as an antibody with a variable heavy chain domain of SEQ ID NO: 22 and a variable light chain domain SEQ ID NO: 21; and/or
an antibody Fab fragment of the antibody binds (i) to human VEGF-A121 with a K_{D} of less than 50 pM as measured by surface plasmon resonance, and (ii) to human IL6 with a K_{D} of less than 50 pM as measured by surface plasmon resonance; and/or
an antibody Fab fragment of the antibody exhibits an aggregation onset temperature of 60° C. or more, in one embodiment 70° C. or more; and/or
an antibody Fab fragment of the antibody exhibits a melting temperature of more than 80° C. as measured by dynamic light scattering.

In another aspect, the invention provides an antibody that binds to human VEGF-A and to human IL6 comprising a VH domain comprising (a) CDR-H1 comprising the amino acid sequence of SEQ ID NO:18, (b) CDR-H2 comprising the amino acid sequence of SEQ ID NO:19, and (c) CDR-H3 comprising the amino acid sequence of SEQ ID NO:20, and a VL domain comprising (d) CDR-LI comprising the amino acid sequence of SEQ ID NO: 15, (e) CDR-L2 comprising the amino acid sequence of SEQ ID NO:16, and (f) CDR-L3 comprising the amino acid sequence of SEQ ID NO: 17, comprising (a) a VH domain comprising an amino acid sequence having at least 85%, 86%, 87%, 88%, 89%, 90%, 91%, 92%, 93%, 94%, 95%, 96%, 97%, 98% or 99% sequence identity to the amino acid sequence of SEQ ID NO:22; and (b) a VL domain comprising an amino acid sequence having at least 85%, 86%, 87%, 88%, 89%, 90%, 91%, 92%, 93%, 94%, 95%, 96%, 97%, 98% or 99% sequence identity to the amino acid sequence of SEQ ID NO:21.

In another aspect, the invention provides an antibody that binds to human VEGF-A and to human IL6 comprising (a) a VH domain comprising an amino acid sequence having at least 85%, 86%, 87%, 88%, 89%, 90%, 91%, 92%, 93%, 94%, 95%, 96%, 97%, 98% or 99% sequence identity to the amino acid sequence of SEQ ID NO:22; and (b) a VL domain comprising an amino acid sequence having at least 85%, 86%, 87%, 88%, 89%, 90%, 91%, 92%, 93%, 94%, 95%, 96%, 97%, 98% or 99% sequence identity to the amino acid sequence of SEQ ID NO:21.

In another aspect, the invention provides an antibody that binds to human VEGF-A and to human IL6 comprising (a) a VH domain comprising an amino acid sequence of SEQ ID NO:22 with up to 15, up to 10, or up to 5 amino acid substitutions; and (b) a variable light chain domain comprising an amino acid sequence of SEQ ID NO: 21 with up to 15, up to 10, or up to 5 amino acid substitutions.

In another aspect, the invention provides an antibody that binds to human VEGF-A and to human IL6 comprising a VH domain comprising (a) CDR-H1 comprising the amino acid sequence of SEQ ID NO:18, (b) CDR-H2 comprising the amino acid sequence of SEQ ID NO: 19, and (c) CDR-H3 comprising the amino acid sequence of SEQ ID NO:20, and a VL domain comprising (d) CDR-LI comprising the amino acid sequence of SEQ ID NO:15, (e) CDR-L2 comprising the amino acid sequence of SEQ ID NO:16, and (f) CDR-L3 comprising the amino acid sequence of SEQ ID NO: 17, comprising (a) a VH domain comprising an amino acid sequence of SEQ ID NO: 22 with up to 15, up to 10, or up to 5 amino acid substitutions; and (b) a variable light chain domain comprising an amino acid sequence of SEQ ID NO:21 with up to 15, up to 10, or up to 5 amino acid substitutions.

In one aspect, the invention provides an antibody that binds to human VEGF-A and to human IL6 comprising a VH domain having at least 90%, 91%, 92%, 93%, 94%, 95%, 96%, 97%, 98%, 99%, or 100% sequence identity to the amino acid sequence of SEQ ID NO:22. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody that binds to human VEGF-A and human IL6 comprising that sequence retains the ability to bind to human VEGF-A and human IL6. In certain aspects, a total of up to 10 amino acids have been substituted, inserted and/or deleted in SEQ ID NO:22. In certain aspects, substitutions, insertions, or deletions occur in regions outside the CDRs (i.e., in the FRs). In a particular aspect, the VH comprises a) CDR-H1 comprising the amino acid sequence of SEQ ID NO: 18, (b) CDR-H2 comprising the amino acid sequence of SEQ ID NO: 19, and (c) CDR-H3 comprising the amino acid sequence of SEQ ID NO:20.

In one aspect, the invention provides an antibody that binds to human VEGF-A and to human IL6 comprising a VL domain having at least 90%, 91%, 92%, 93%, 94%, 95%, 96%, 97%, 98%, 99%, or 100% sequence identity to the amino acid sequence of SEQ ID NO:21. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody that binds to human VEGF-A and human IL6 comprising that sequence retains the ability to bind to human VEGF-A and human IL6. In certain aspects, a total of up to 10 amino acids have been substituted, inserted and/or deleted in SEQ ID NO:21. In certain aspects, substitutions, insertions, or deletions occur in regions outside the CDRs (i.e., in the FRs). In a particular aspect, the VL comprises (d) CDR-LI comprising the amino acid sequence of SEQ ID NO:15, (e) CDR-L2 comprising the amino acid sequence of SEQ ID NO: 16, and (f) CDR-L3 comprising the amino acid sequence of SEQ ID NO: 17.

In another aspect, an antibody that binds to human VEGF-A and human IL6 is provided, wherein the antibody comprises a VH sequence as in any of the aspects provided above, and a VL sequence as in any of the aspects provided above. In one aspect, the antibody comprises the VH and VL sequences SEQ ID NO:22 and SEQ ID NO: 21, respectively, including post-translational modifications of those sequences.

In another aspect, an antibody that binds to human VEGF-A and human IL6 is provided, wherein the antibody comprises a heavy chain amino acid sequence of SEQ ID NO:24 and a light chain amino acid sequence of SEQ ID NO:23.

In a further aspect of the invention, an antibody that binds to human VEGF-A and human IL6 according to any of the above aspects is a monoclonal antibody. In one aspect, an antibody that binds to human VEGF-A and human IL6 is an antibody fragment, e.g., a Fv, Fab, Fab′, scFv, diabody, or F (ab) 2 fragment. In another aspect, the antibody is a full length antibody.

In another aspect the invention provides an antibody that binds to IL6 that is derived from an antibody of the invention. The IL6 paratope disclosed for antibodies of the invention may be used to provide further antibodies, e.g. monospecific antibodies or bispecific antibodies that bind to IL6 and another antigen. The IL6 paratope of antibody 6HVL4.1 as disclosed herein was identified via x-ray crystallography (Example 13). Antibody 6HVL4.1 is based on a VH domain having a human VH3 framework and a VL domain having a human Vkappal framework. Antibodies comprising the IL6 paratope of antibody 6HVL4.1 bind to the same epitope on IL6. All embodiments disclosed herein for antibodies of the invention that bind to human VEGF-A and human IL6 apply for antibodies that bind to IL6 as well.

Hence, in one embodiment the invention provides an antibody that binds to human IL6 comprising;
c) a VH domain based on a human VH3 framework, wherein the IL6 paratope comprises amino acid residues Y1, I2, Q3, Y26, E27, F28, T29, H30, Q31, D32, P52a, R94, 196, D97, F98, D101, T102, and a VL domain based on a human Vkappal framework, wherein the IL6 paratope comprises amino acid residues Y49, D50, S53, N54, Y55, P56, S57, Y91, Y96; or
d) a VH domain based on a human VH3 framework, wherein the IL6 paratope comprises amino acid residues Y1, P2, Q3, V26, L27, F28, K29, H30, Q31, D32, P52a, R94, L96, D97, F98, D101, E102, and a VL domain based on a human Vkappal framework, wherein the IL6 paratope comprises amino acid residues Y49, D50, D53, R54, Y55, P56, E57, Y91, Y96 (numbering according to Kabat).

In another aspect the invention provides an antibody that binds to IL6 that binds to the same epitope on IL6 as an antibody with a VL domain of SEQ ID NO: 35 and a VH domain of SEQ ID NO: 36. In one embodiment the antibody comprises a VH domain having a human VH3 framework, wherein the IL6 paratope comprises amino acid residues 1, 2, 3, 26, 27, 28, 29, 30, 31, 32, 52a, 94, 96, 97, 98, 101, 102 of an antibody that binds to human VEGF-A and IL6 of the invention and a VL domain having a human Vkappal framework, wherein the IL6 paratope comprises amino acid residues 49, 50, 53, 54, 55, 56, 57, 91, 96 of an antibody that binds to human VEGF-A and IL6 of the invention.

In one embodiment, said antibody that binds to IL6 as described above, is a multispecific antibody that binds to IL6 and another target.

In a further aspect, an antibody that binds to human VEGF-A and human IL6 according to any of the above aspects or an antibody that binds to human IL6 according to any of the above aspects may incorporate any of the features, singly or in combination, as described in Sections 1-5 below:

### 1. Antibody Affinity

In certain embodiments, an antibody provided herein binds to VEGF-A with a dissociation constant (KD) of ≤1 nM, ≤0.1 nM, or ≤0.01 nM. In a preferred embodiment an antibody provided herein binds to human VEGF-A with a dissociation constant (KD) of ≤10 pM, in a preferred embodiment≤5 pM. In a preferred embodiment an antibody provided herein binds to human VEGFA-121 with a dissociation constant (KD) of ≤10 pM, in a preferred embodiment≤5 pM. In a preferred embodiment an antibody provided herein binds to human VEGFA-165 with a dissociation constant (KD) of ≤10 pM, in a preferred embodiment≤5 pM.

In certain embodiments, an antibody that binds to IL6 has a dissociation constant (KD) of ≤1 nM, ≤0.1 nM, or ≤0.03 nM. In a preferred embodiment an antibody provided herein binds to human IL6 with a dissociation constant (Kp) of ≤10 pM, in a preferred embodiment≤5 pM. In one aspect, K_{D} is measured using a surface plasmon resonance assay, in one embodiment a BIACORE® surface plasmon resonance assay.

In another aspect, K_{D} is measured using a KinExA assay. In one embodiment, Kp is measured using a KinExA assay under the conditions as described below in the Materials & general methods section for either detection of K_{D} of VEGF-A binding or detection of K_{D} of IL6 binding.

For example, the K_{D} of antibody binding to VEGF-A is measured in an assay using a KinExA 3200 instrument from Sapidyne Instruments (Boise, ID), PMMA beads are coated with antigen according to the KinExA Handbook protocol (Adsorption coating, Sapidyne) using 30 μg of Anti-VEGF-Antibody MAB293 (R&D) in 1 ml PBS (pH7.4). KinExA equilibrium assay is performed at room temperature using PBS pH 7.4 with 0.01% BSA and 0.01% Tween20 as running buffer, samples and beads are prepared in LowCross buffer (Candor Bioscience). A flow rate of 0.25 ml/min is used. A constant amount of VEGFA-121-His (50 pM and in a second experiment 500 pM) is titrated with the tested antibody and equilibrated mixtures are drawn through a column of anti-VEGF antibody (Mab293) coupled beads in the KinExA system at a volume of 750 μl for 50 pM constant VEGF and at a volume of 125 μl for 500 pM constant VEGF. Detection of bound VEGFA-121 is done using a second biotinylated anti-VEGF antibody (BAF293) with a concentration of 250 ng/ml and following by an injection of 250 ng/ml Streptavidin Alexa Fluor™ 647 conjugate in sample buffer. The K_{D} is obtained from non-linear regression analysis of the data using a one-site homogeneous binding model contained within the KinExA software (Version 4.0.11) using the “standard analysis” method. The software calculates the K_{D} and determines the 95% confidence interval by fitting the data points to a theoretical K_{D} curve. The 95% confidence interval is given as Kp low and K_{D} high.

For example, the K_{D} of antibody binding to IL6 is measured in an assay using surface plasmon resonance (SPR) on a Biacore 8K instrument (Cytiva) at 25° C. using HBS-EP+ (1×; BR100669; Cytiva) as running buffer. A Human Fab Binder (28958325, Cytiva) is diluted at a final concentration of 10 μg/ml in 10 mM sodium acetate buffer, pH 5.0 and immobilized on a CM5 sensor chip using standard amine coupling chemistry. Prior to the protein measurements five startup cycles are optionally performed for conditioning purposes, wherein on each cycle HBS-EP+ buffer is flown for about 120 s followed by the regeneration of the derivatized chip surface by applying 10 mM Glycine buffer pH2.0 for 60 s. Antibody Fab fragment at a concentration of 75 nM is captured on this surface for 60 s at a flow rate of 10 μl/min in HBS-EP+ buffer. No Fab fragment is applied to the reference channel. Subsequently, human or cynomolgus monkey IL-6 are applied in an appropriate dilution series in HBS-EP+ buffer at a flow rate of 30 μl/min (preferably using a contact time of 180 s and a dissociation time of 720 s). Regeneration of the derivatized chip surface was achieved is described above. Data is evaluated using 8K Evaluation software (Biacore Insight Evaluation 3.0).

### 2. Antibody Fragments

In certain aspects, an antibody provided herein is an antibody fragment.

In one aspect, the antibody fragment is a Fab, Fab′, Fab-SH, or F (ab) 2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab-SH are Fab′ fragments in which the cysteine residue(s) of the constant domains bear a free thiol group. Pepsin treatment yields an F(ab′) 2 fragment that has two antigen-binding sites (two Fab fragments) and a part of the Fc region. For discussion of Fab and F(ab′) 2 fragments comprising salvage receptor binding epitope residues and having increased in vivo half-life, see U.S. Pat. No. 5,869,046.

Antibody fragments can be made by various techniques, including but not limited to proteolytic digestion of an intact antibody as well as recombinant production by recombinant host cells (e.g., E. coli, CHO), as described herein.

In a preferred embodiment the antibody provided herein is a Fab fragment.

In one embodiment the VH domain of the antibody provided herein comprises a human VH3 framework.

In one embodiment the VL domain of the antibody provided herein comprises a human Vkappal framework.

In one embodiment the CL domain of the antibody provided herein is of kappa isotype.

In one embodiment the CH1 domain of the antibody provided herein is of human IgG1 isotype.

In a preferred embodiment, the antibody provided herein is a Fab fragment comprising a CL domain of kappa isotype and a CH1 domain of human IgG1 isotype.

3. Thermal stability

Antibodies provided herein exhibit superior thermal stability. In certain embodiments, a Fab fragment of an antibody provided herein exhibits an aggregation onset temperature of 60° C. or more, in one embodiment 70° C. or more. In certain embodiments, a Fab fragment of an antibody provided herein exhibits a melting temperature of more than 80° C. as measured by dynamic light scattering. 4. Multispecific Antibodies

In certain aspects, an antibody provided herein is a multispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities.

Multispecific antibodies with three or more binding specificities comprising antibodies provided herein may be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). Various further molecular formats for multispecific antibodies are known in the art and are included herein (see e.g., Spiess et al., Mol Immunol 67 (2015) 95-106). 5. Antibody Variants

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain aspects, antibody variants having one or more amino acid substitutions are provided. Sites of interest for substitutional mutagenesis include the CDRs and FRs. Conservative substitutions are shown in the Table below under the heading of “preferred substitutions”. More substantial changes are provided in Table 1 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Th |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties;
(1) hydrophobic: Norleucine, Met, Ala, Val, Leu, Ile;
(2) neutral hydrophilic: Cys, Ser, Thr, Asn, Gln;
(3) acidic: Asp, Glu;
5 (4) basic: His, Lys, Arg;
(5) residues that influence chain orientation: Gly. Pro;
(6) aromatic: Trp, Tyr, Phe.

Non-conservative substitutions will entail exchanging a member of one of these classes for a member of another class.

One type of substitutional variant involves substituting one or more CDR residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity-matured antibody, which may be conveniently generated, e.g., using phage display-based affinity maturation techniques such as those described herein. Briefly, one or more CDR residues are mutated and the variant antibodies displayed on phage and screened for a particular biological activity (e.g., binding affinity).

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

a) Glycosylation variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e.g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8. knockout CHO cells (see, e.g., Yamane-Ohnuki et al. Biotech. Bioeng. 87:614-622 (2004); Kanda, Y. et al., Biotechnol. Bioeng., 94 (4); 680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342: WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### b) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Natl Acad. Sci. USA 83:7059-7063 (1986)) and Hellstrom, I et al., Proc. Natl Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 968 non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18 (12); 1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9 (2); 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half lives and improved binding to the neonatal Fc receptor (FcRn), which is responsible for the transfer of maternal IgG₃ to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues I253, H310, H433, N434, and H435 (EU index numbering) are involved in the interaction (Medesan, C., et al., Eur. J. Immunol. 26 (1996) 2533: Firan, M., et al., Int. Immunol. 13 (2001) 993: Kim, J. K., et al., Eur. J. Immunol. 24 (1994) 542). Residues I253, H310, and H435 were found to be critical for the interaction of human Fc with murine FcRn (Kim, J. K., et al., Eur. J. Immunol. 29 (1999) 2819). Studies of the human Fc-human FcRn complex have shown that residues I253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG₁ Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG₁ Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

c) Cysteine engineered antibody variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### B. Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In one aspect, isolated nucleic acids encoding an antibody of the invention are provided.

In one aspect, a method of making an antibody that binds to human VEGF-A and human IL6 is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an antibody that binds to human VEGF-A and human IL6, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified. In one embodiment the host cell is an E. coli cell.

Vertebrate cells may also be used as hosts. For example, mammalian cell lines that are adapted to grow in suspension may be useful. Other examples of useful mammalian host cell lines are monkey kidney CVI line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK: buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Nat'l. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell). In one preferred embodiment the host cell is a CHO cell. Production of antibodies of the invention in CHO cells may improve syringeability of the antibody.

### C. Pharmaceutical Compositions

In a further aspect, provided are pharmaceutical compositions comprising any of the antibodies provided herein, e.g., for use in any of the below therapeutic methods. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions of an antibody that binds to human VEGF-A and human IL6 as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride: hexamethonium chloride: benzalkonium chloride: benzethonium chloride: phenol, butyl or benzyl alcohol: alkyl parabens such as methyl or propyl paraben: catechol: resorcinol: cyclohexanol: 3-pentanol; and m-cresol); low molecular weight (less than about 10 residues) polypeptides: proteins, such as serum albumin, gelatin, or immunoglobulins: hydrophilic polymers such as polyvinylpyrrolidone: amino acids such as glycine, glutamine, asparagine, histidine, arginine, or lysine: monosaccharides, disaccharides, and other carbohydrates including glucose, mannose, or dextrins; chelating agents such as EDTA: sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH2O, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

The pharmaceutical composition herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the antibody, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

### D. Therapeutic Methods and Routes of Administration

Any of the antibodies that bind to human VEGF-A and human IL6 provided herein may be used in therapeutic methods.

In one aspect, an antibody that binds to human VEGF-A and human IL6 for use as a medicament is provided. In further aspects, an antibody that binds to human VEGF-A and human IL6 for use in treating a vascular disease is provided. In certain aspects, an antibody that binds to human VEGF-A and human IL6 for use in a method of treatment is provided. In certain aspects, the invention provides an antibody that binds to human VEGF-A and human IL6 for use in a method of treating an individual having a vascular disease comprising administering to the individual an effective amount of the antibody that binds to human VEGF-A and human IL6. In one such aspect, the method further comprises administering to the individual an effective amount of at least one additional therapeutic agent (e.g., one, two, three, four, five, or six additional therapeutic agents), e.g., as described below. In further aspects, the invention provides an antibody that binds to human VEGF-A and human IL6 for use in inhibiting angiogenesis. In certain aspects, the invention provides an antibody that binds to human VEGF-A and human IL6 for use in a method inhibiting angiogenesis in an individual comprising administering to the individual an effective amount of the antibody that binds to human VEGF-A and human IL6 to inhibit angiogenesis. An “individual” according to any of the above aspects is preferably a human.

In further aspects, an antibody that binds to human VEGF-A and human IL6 for use in treating an ocular disease is provided. In one embodiment the ocular disease is selected from AMD (in one embodiment wet AMD, dry AMD, intermediate AMD, advanced AMD, and geographic atrophy (GA)), macular degeneration, macular edema, DME (in one embodiment focal, non-center DME and diffuse, center-involved DME), retinopathy, diabetic retinopathy (DR) (in one embodiment proliferative DR (PDR), non-proliferative DR (NPDR), and high-altitude DR), other ischemia-related retinopathies, ROP, retinal vein occlusion (RVO) (in one embodiment central (CRVO) and branched (BRVO) forms), CNV (in one embodiment myopic CNV), corneal neovascularization, diseases associated with corneal neovascularization, retinal neovascularization, diseases associated with retinal/choroidal neovascularization, central serous retinopathy (CSR), pathologic myopia, von Hippel-Lindau disease, histoplasmosis of the eye, FEVR, Coats' disease, Norrie Disease, retinal abnormalities associated with osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, including but not limited to CMV retinitis, ocular melanoma, retinal blastoma, conjunctivitis (in one embodiment infectious conjunctivitis and non-infectious (in one embodiment allergic) conjunctivitis), Leber congenital amaurosis (also known as Leber's congenital amaurosis or LCA), uveitis (including infectious and non-infectious uveitis), choroiditis (in one embodiment multifocal choroiditis), ocular histoplasmosis, blepharitis, dry eye, traumatic eye injury, Sjögren's disease, and other ophthalmic diseases wherein the disease or disease is associated with ocular neovascularization, vascular leakage, and/or retinal edema or retinal atrophy. In one embodiment the ocular disease is selected from AMD (in one embodiment wet AMD, dry AMD, intermediate AMD, advanced AMD, and geographic atrophy (GA)), macular degeneration, macular edema, DME (in one embodiment focal, non-center DME and diffuse, center-involved DME), retinopathy, diabetic retinopathy (DR) (in one embodiment proliferative DR (PDR), non-proliferative DR (NPDR), and high-altitude DR.

In a further aspect, the invention provides for the use of an antibody that binds to human VEGF-A and human IL6 in the manufacture or preparation of a medicament. In one aspect, the medicament is for treatment of a vascular disease. In a further aspect, the medicament is for use in a method of treating a vascular disease comprising administering to an individual having a vascular disease an effective amount of the medicament. In one such aspect, the method further comprises administering to the individual an effective amount of at least one additional therapeutic agent, e.g., as described below.

In one aspect, the medicament is for treatment of an ocular disease. In a further aspect, the medicament is for use in a method of treating an ocular disease comprising administering to an individual having an ocular disease an effective amount of the medicament. In one such aspect, the method further comprises administering to the individual an effective amount of at least one additional therapeutic agent, e.g., as described below.

In a further aspect, the invention provides a method for treating a vascular disease. In one aspect, the method comprises administering to an individual having such vascular disease an effective amount of an antibody that binds to human VEGF-A and human IL6. In one such aspect, the method further comprises administering to the individual an effective amount of at least one additional therapeutic agent, as described below.

In a further aspect, the invention provides a method for treating an ocular disease. In one aspect, the method comprises administering to an individual having such ocular disease an effective amount of an antibody that binds to human VEGF-A and human IL6. In one such aspect, the method further comprises administering to the individual an effective amount of at least one additional therapeutic agent, as described below.

An “individual” according to any of the above aspects may be a human.

In a further aspect, the invention provides pharmaceutical compositions comprising any of the antibodies that bind to human VEGF-A and human IL6 provided herein, e.g., for use in any of the above therapeutic methods. In one aspect, a pharmaceutical composition comprises any of the antibodies that bind to human VEGF-A and human IL6 provided herein and a pharmaceutically acceptable carrier. In another aspect, a pharmaceutical composition comprises any of the antibodies that bind to human VEGF-A and human IL6 provided herein and at least one additional therapeutic agent, e.g., as described below.

The antibody of the invention may be administered by intravitreal administration (e.g., intravitreal injection) or using a port delivery device. In one embodiment the antibody of the invention is administered using a port delivery device over a period of six months or more, in one embodiment 8 months or more, in one embodiment 9 months or more, in one embodiment 12 months or more, before the port delivery device is refilled. In one embodiment the antibody of the invention is administered using a port delivery device, wherein the antibody is applied into the port delivery device at a concentration of 150 mg/ml or more, in one embodiment at a concentration of 200 mg/ml or more.

Antibodies of the invention can be administered alone or used in a combination therapy. For instance, the combination therapy includes administering an antibody of the invention and administering at least one additional therapeutic agent (e.g. one, two, three, four, five, or six additional therapeutic agents).

In certain embodiments according to (or as applied to) any of the embodiments above, the ocular disorder is an intraocular neovascular disease selected from the group consisting of proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, retinal vein occlusion (RVO), including CRVO and BRVO, corneal neovascularization, retinal neovascularization, and retinopathy of prematurity (ROP).

In some instances, an antibody that binds to human VEGF-A and human IL6 provided herein may be administered in combination with at least one additional therapeutic agent for treatment of an ocular disorder, for example, an ocular disorder described herein (e.g., AMD (e.g., wet AMD), DME, DR, RVO, or GA).

Any suitable AMD therapeutic agent can be administered as an additional therapeutic agent in combination with an antibody that binds to human VEGF and human IL6 as provided herein for treatment of an ocular disorder (e.g., AMD, DME, DR, RVO, or GA), including, but not limited to, a VEGF antagonist, for example, an anti-VEGF antibody (e.g., LUCENTIS® (ranibizumab), RTH-258 (formerly ESBA-1008, an anti-VEGF single-chain antibody fragment: Novartis), or a bispecific anti-VEGF antibody (e.g., an anti-VEGF/anti-angiopoeitin 2 bispecific antibody such as faricimab; Roche)), a soluble VEGF receptor fusion protein (e.g., EYLEA® (aflibercept)), an anti-VEGF DARPin® (e.g., abicipar pegol; Molecular Partners AG/Allergan), or an anti-VEGF aptamer (e.g,. MACUGEN® (pegaptanib sodium)); a platelet-derived growth factor (PDGF) antagonist, for example, an anti-PDGF antibody, an anti-PDGFR antibody (e.g., REGN2176-3), an anti-PDGF-BB pegylated aptamer (e.g., FOVISTA®; Ophthotech/Novartis), a soluble PDGFR receptor fusion protein, or a dual PDGF/VEGF antagonist (e.g., a small molecule inhibitor (e.g., DE-120 (Santen) or X-82 (TyrogeneX)) or a bispecific anti-PDGF/anti-VEGF antibody)); VISUDYNE® (verteporfin) in combination with photodynamic therapy; an antioxidant; a complement system antagonist, for example, a complement factor C5 antagonist (e.g., a small molecule inhitor (e.g., ARC-1905; Opthotech) or an anti-C5 antibody (e.g., LFG-316; Novartis), a properdin antagonist (e.g., an anti-properdin antibody, e.g., CLG-561: Alcon), or a complement factor D antagonist (e.g., an anti-complement factor D antibody, e.g., lampalizumab; Roche)); a C3 blocking peptide (e.g., APL-2, Appellis); a visual cycle modifier (e.g., emixustat hydrochloride); squalamine (e.g., OHR-102: Ohr Pharmaceutical); vitamin and mineral supplements (e.g., those described in the Age-Related Eye Disease Study 1 (AREDSI; zinc and/or antioxidants) and Study 2 (AREDS2; zinc, antioxidants, lutein, zeaxanthin, and/or omega-3 fatty acids)); a cell-based therapy, for example, NT-501 (Renexus); PH-05206388 (Pfizer), huCNS-SC cell transplantation (StemCells), CNTO-2476 (umbilical cord stem cell line: Janssen), OpRegen (suspension of RPE cells; Cell Cure Neurosciences), or MA09-hRPE cell transplantation (Ocata Therapeutics); a tissue factor antagonist (e.g., hl-con1; Iconic Therapeutics); an alpha-adrenergic receptor agonist (e.g,. brimonidine tartrate; Allergan); a peptide vaccine (e.g., S-646240; Shionogi); an amyloid beta antagonist (e.g., an anti-beta amyloid monoclonal antibody, e.g., GSK-933776); an SIP antagonist (e.g., an anti-SIP antibody, e.g., iSONEP™; Lpath Inc); a ROBO4 antagonist (e.g., an anti-ROBO4 antibody, e.g., DS-7080a; Daiichi Sankyo); a lentiviral vector expressing endostatin and angiostatin (e.g., RetinoStat); and any combination thereof. In some instances, AMD therapeutic agents (including any of the preceding AMD therapeutic agents) can be co-formulated. For example, the anti-PDGFR antibody REGN2176-3 can be co-formulated with aflibercept (EYLEAR). In some instances, such a co-formulation can be administered in combination with an antibody that binds to human VEGF and human IL6 of the invention. In some instances, the ocular disorder is AMD (e.g., wet AMD).

Any suitable DME and/or DR therapeutic agent can be administered in combination with an antibody that binds to human VEGF and human IL6 of the invention for treatment of an ocular disorder (e.g., AMD, DME, DR, RVO, or GA), including, but not limited, to a VEGF antagonist (e.g., LUCENTIS® or EYLEA®), a corticosteroid (e.g., a corticosteroid implant (e.g., OZURDEX® (dexamethasone intravitreal implant) or ILUVIEN® (fluocinolone acetonide intravitreal implant)) or a corticosteroid formulated for administration by intravitreal injection (e.g., triamcinolone acetonide)), or combinations thereof. In some instances, the ocular disorder is DME and/or DR.

An antibody that binds to human VEGF and human IL6 as provided herein may be administered in combination with a therapy or surgical procedure for treatment of an ocular disorder (e.g., AMD, DME, DR, RVO, or GA), including, for example, laser photocoagulation (e.g., panretinal photocoagulation (PRP)), drusen lasering, macular hole surgery, macular translocation surgery, implantable miniature telescopes, PHI-motion angiography (also known as micro-laser therapy and feeder vessel treatment), proton beam therapy, microstimulation therapy, retinal detachment and vitreous surgery, scleral buckle, submacular surgery, transpupillary thermotherapy, photosystem I therapy, use of RNA interference (RNAi), extracorporeal rheopheresis (also known as membrane differential filtration and rheotherapy), microchip implantation, stem cell therapy, gene replacement therapy, ribozyme gene therapy (including gene therapy for hypoxia response element, Oxford Biomedica; Lentipak, Genetix; and PDEF gene therapy, GenVec), photoreceptor/retinal cells transplantation (including transplantable retinal epithelial cells, Diacrin, Inc.; retinal cell transplant, e.g., Astellas Pharma US, Inc., ReNeuron, CHA Biotech), acupuncture, and combinations thereof.

Such combination therapies noted above encompass combined administration (where two or more therapeutic agents are included in the same or separate formulations), and separate administration, in which case, administration of the antibody that binds to human VEGF and human IL6 of the inventioncan occur prior to, simultaneously, and/or following, administration of the additional therapeutic agent or agents. In one embodiment, administration of the antibody that binds to human VEGF and human IL6 of the invention and administration of an additional therapeutic agent occur within about one, two, three, four, or five months, or within about one, two or three weeks, or within about one, two, three, four, five, or six days, of each other.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0. 1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg. 2.0 mg/kg, 4.0 mg/kg or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses may be administered. The progress of this therapy is easily monitored by conventional techniques and assays.

### E. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, vials, syringes, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice.

Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### F. Devices

The antibody of the invention may be administered into the eye using an ocular implant, in one embodiment using a port delivery device.

A port delivery device is an implantable, refillable device that can release a therapeutic agent (e.g., an antibody of the invention) over a period of months (e.g., 1, 2, 3, 4, 5, 6, 7, 8, 9, 10, 11, 12, or more months). Exemplary port delivery devices that may be used include those from ForSight Labs, LLC and/or ForSight VISION4, for example, as described in International Patent Application Publication Nos. WO 2010/088548, WO2015/085234, WO 2013/116061, WO 2012/019176, WO 2013/040247, and WO 2012/019047, which are incorporated herein by reference in their entirety.

For example, the invention provides port delivery devices that include reservoirs containing any of the antibodies described herein. The port delivery device may further include a proximal region, a tubular body coupled to the proximal region in fluid communication with the reservoir, and one or more outlets in fluid communication with the reservoir and configured to release the composition into the eye. The tubular body may have an outer diameter configured to be inserted through an incision or opening in the eye of about 0.5 mm or smaller. The device may be about 1 mm to about 15 mm in length (e.g., about 1 mm, about 2 mm, about 4 mm, about 5 mm, about 6 mm, about 7 mm, about 9 mm, about 11 mm, about 13 mm, or about 15 mm in length). The reservoir may have any suitable volume. In some instances, the reservoir has a volume of about 1 μl to about 100 μl (e.g., about 1 μl, about 5 μl, about 10 μl, about 20 μl, about 50 μl, about 75 μl, or about 100 μl). The device or its constituent parts may be made of any suitable material, for example, polyimide.

In some instances, the port delivery device includes a reservoir containing any of the antibodies described herein and one or more additional compounds.

In some instances, the port delivery device includes any of the antibodies or antibody conjugates described herein and an additional VEGF antagonist.

### 3. Specific Embodiments of the Invention

In the following specific embodiments of the invention are listed.
1. An antibody that binds to human VEGF-A and to human IL6 comprising a VH domain comprising (a) CDR-H1 comprising the amino acid sequence of SEQ ID NO: 18, (b) CDR-H2 comprising the amino acid sequence of SEQ ID NO: 19, and (c) CDR-H3 comprising the amino acid sequence of SEQ ID NO:20, and a VL domain comprising (d) CDR-LI comprising the amino acid sequence of SEQ ID NO: 15, (e) CDR-L2 comprising the amino acid sequence of SEQ ID NO:16, and (f) CDR-L3 comprising the amino acid sequence of SEQ ID NO:17, comprising (a) a VH domain comprising an amino acid sequence having at least 85%, 86%, 87%, 88%, 89%, 90%, 91%, 92%, 93%, 94%, 95%, 96%, 97%, 98% or 99% sequence identity to the amino acid sequence of SEQ ID NO:22; and (b) a VL domain comprising an amino acid sequence having at least 85%, 86%, 87%, 88%, 89%, 90%, 91%, 92%, 93%, 94%, 95%, 96%, 97%, 98% or 99% sequence identity to the amino acid sequence of SEQ ID NO:21.
2. An antibody that binds to human VEGF-A and to human IL6 comprising a VH domain comprising (a) CDR-H1 comprising the amino acid sequence of SEQ ID NO: 18, (b) CDR-H2 comprising the amino acid sequence of SEQ ID NO: 19, and (c) CDR-H3 comprising the amino acid sequence of SEQ ID NO:20, and a VL domain comprising (d) CDR-LI comprising the amino acid sequence of SEQ ID NO: 15, (e) CDR-L2 comprising the amino acid sequence of SEQ ID NO:16, and (f) CDR-L3 comprising the amino acid sequence of SEQ ID NO:17, comprising a variable heavy chain domain comprising an amino acid sequence of SEQ ID NO:22 with up to 5 amino acid substitutions; and a variable light chain domain comprising an amino acid sequence of SEQ ID NO:21 with up to 5 amino acid substitutions.
3. An antibody that binds to human VEGF-A and to human IL6 comprising (a) a VH domain comprising an amino acid sequence of SEQ ID NO:22 with up to 15, up to 10, or up to 5 amino acid substitutions; and (b) a variable light chain domain comprising an amino acid sequence of SEQ ID NO:21 with up to 15, up to 10, or up to 5 amino acid substitutions.
4. An antibody that binds to human VEGF-A and to human IL6, comprising a VH sequence of SEQ ID NO:22 and a VL sequence of SEQ ID NO:21.
5. The antibody of one of the preceding embodiments, comprising a heavy chain amino acid sequence of SEQ ID NO:24 and a light chain amino acid sequence of SEQ ID NO:23.
6. The antibody of any one of the preceding embodiments, wherein the VEGF-A paratope comprises amino acid residues from CDR-H2, CDR-LI and CDR-L3 of the antibody, wherein the IL6 paratope comprises amino acid residues from the CDR-H1, CDR-H3 and CDR-L2 of the antibody or wherein the IL6 paratope comprises amino acid residues from CDR-H2, CDR-LI and CDR-L3 of the antibody, wherein the VEGF-A paratope comprises amino acid residues from the CDR-H1, CDR-H3 and CDR-L2 of the antibody; and/or
   the pair of the variable light chain domain and the variable heavy chain domain simultaneously binds to human VEGF-A and human IL6; and/or
   the antibody binds to the same epitope on human VEGF-A and to the same epitope on human IL6 as an antibody with a variable heavy chain domain of SEQ ID NO: 22 and a variable light chain domain SEQ ID NO: 21; and/or
   an antibody Fab fragment of the antibody binds (i) to human VEGF-A121 with a K_{D} of less than 50 pM as measured by surface plasmon resonance, and (ii) to human IL6 with a K_{D} of less than 50 pM as measured by surface plasmon resonance; and/or
   an antibody Fab fragment of the antibody exhibits an aggregation onset temperature of 60° C. or more, in one embodiment 70° C. or more; and/or
   an antibody Fab fragment of the antibody exhibits a melting temperature of more than 80° C. as measured by dynamic light scattering.
7. An antibody that specifically binds to human VEGF-A and to human IL6, comprising a heavy chain amino acid sequence of SEQ ID NO:24 and a light chain amino acid sequence of SEQ ID NO:23.
8. The antibody of any one of the preceding embodiments, wherein the antibody is a Fab fragment.
9. The antibody of any one of the preceding embodiments, wherein the antibody is a bispecific antibody fragment.
10. The antibody of any one of the preceding embodiments, which is a monoclonal antibody.
11. The antibody of any one of the preceding embodiments, wherein an antibody Fab fragment of the antibody exhibits an aggregation onset temperature of 70° C. and more.
12. The antibody of any one of the preceding embodiments, wherein an antibody Fab fragment of the antibody exhibits a melting temperature of more than 80° C. as measured by dynamic light scattering.
13. The antibody of any one of the preceding embodiments, which is a monoclonal antibody.
14. The antibody of any one of the preceding embodiments, which is an antibody fragment that binds to human VEGF-A and to human IL6.
15. The antibody of any one of the preceding embodiments, wherein the antibody is bispecific.
16. The antibody of any one of the preceding embodiments, wherein the antibody is a Fab fragment.
17. The antibody of any one of the preceding embodiments, wherein the antibody is a bispecific antibody fragment.
18. The antibody of any one of the preceding embodiments, wherein the antibody is a multispecific antibody.
19. The antibody of any one of the preceding embodiments, wherein the antibody specifically binds to human VEGF-A.
20. The antibody of any one of the preceding embodiments, wherein the antibody specifically binds to human IL6.
21. An antibody that binds to human IL6 that binds to the same epitope on IL6 as an antibody with a VL domain of SEQ ID NO: 35 and a VH domain of SEQ ID NO: 36.
22. An antibody that binds to human IL6, wherein the antibody comprises a VH domain having a human VH3 framework, wherein the IL6 paratope comprises amino acid residues 1, 2, 3, 26, 27, 28, 29, 30, 31, 32, 52a, 94, 96, 97, 98, 101, 102 of an antibody that binds to human VEGF-A and IL6 according to any one of embodiments 1 to 20 and a VL domain having a human Vkappal framework, wherein the IL6 paratope comprises amino acid residues 49, 50, 53, 54, 55, 56, 57, 91, 96 of an antibody that binds to human VEGF-A and IL6 according to any one of embodiments 1 to 20.
23. An antibody that binds to human IL6 comprising;
   a) a VH domain based on a human VH3 framework, wherein the IL6 paratope comprises amino acid residues Y1, I2, Q3, Y26, E27, F28, T29, H30, Q31, D32, P52a, R94, 196, D97, F98, D101, T102, and a VL domain based on a human Vkappal framework, wherein the IL6 paratope comprises amino acid residues Y49, D50, S53, N54, Y55, P56, S57, Y91, Y96; or
   b) a VH domain based on a human VH3 framework, wherein the IL6 paratope comprises amino acid residues Y1, P2, Q3, V26, L27, F28, K29, H30, Q31, D32, P52a, R94, L96, D97, F98, D101, E102, and a VL domain based on a human Vkappal framework, wherein the IL6 paratope comprises amino acid residues Y49, D50, D53, R54, Y55, P56, E57, Y91, Y96 (numbering according to Kabat).
24. An isolated nucleic acid encoding the antibody of any of embodiments 1 to 23.
25. A host cell comprising the nucleic acid of embodiment 24.
26. A method of producing an antibody that binds to human VEGF-A and to human IL6 comprising culturing the host cell of embodiment 25 so that the antibody is produced.
27. The method of embodiment 26, wherein the host cell is a CHO cell.
28. A pharmaceutical formulation comprising the antibody of any one of embodiments 1 to 23 and a pharmaceutically acceptable carrier.
29. A port delivery device comprising the antibody of any one of embodiments 1 to 23.
30. The antibody of any one of embodiments 1 to 23 for use as a medicament.
31. The method of embodiment 26, further comprising recovering the antibody from the host cell.
32. An antibody produced by the method of embodiment 26 or 31.
33. A pharmaceutical formulation comprising the antibody of any one of embodiments 1 to 23 and a pharmaceutically acceptable carrier.
34. The antibody of any one of embodiments 1 to 23 for use as a medicament.
35. The antibody of any one of embodiments 1 to 23 for use in the treatment of a vascular disease.
36. The antibody of any one of embodiments 1 to 23 for use in the treatment of an ocular vascular disease.
37. Use of the antibody of any one of embodiments 1 to 23 or the pharmaceutical composition of embodiment 65 in the manufacture of a medicament.
38. Use of the antibody of any one of embodiments 1 to 23 or the pharmaceutical composition of embodiment 65 in the manufacture of a medicament for inhibiting angiogenesis.
39. A method of treating an individual having a vascular disease comprising administering to the individual an effective amount of the antibody of one of embodiments 1 to 23 or the pharmaceutical formulation of embodiment 33.
40. A method of treating an individual having an ocular vascular disease comprising administering to the individual an effective amount of the antibody of one of embodiments 1 to 23 or the pharmaceutical formulation of embodiment 33.
41. A method of inhibiting angiogenesis in an individual comprising administering to the individual an effective amount of the antibody of any of embodiments 1 to 23 or the pharmaceutical formulation of embodiment 33 to inhibit angiogenesis.
42. A port delivery device comprising the antibody of any of embodiments 1 to 23 or the pharmaceutical formulation of embodiment 33.
43. The antibody of any of embodiments 1 to 23 or the pharmaceutical formulation of embodiment 33 for ocular administration by a port delivery device.
44. The antibody of any of embodiments 1 to 23 or the pharmaceutical formulation of embodiment 33 for ocular administration by a port delivery device according to embodiment 42, wherein the administration is over a period of six months or more, in one embodiment 8 months or more, in one embodiment 9 months or more, before the port delivery device is refilled.
45. The of any of embodiments 1 to 23 or the pharmaceutical formulation of embodiment 33 for use as a medicament by administrating the antibody or the pharmaceutical formulation using a port delivery device, wherein the antibody is applied into the port delivery device at a concentration of 150 mg/ml or more, in one embodiment at a concentration of 200 mg/ml or more.

DESCRIPTION OF THE AMINO ACID SEQUENCES

| | |
|---|---|
| SEQ ID NO: 1 | VL VH6L-BM (disclosed in WO2012/163520) |
| | NIQKTQSPSSLSASVGDRVTITCRALWHISSYLAWYQQKPGKAPKL |
| | LIYAASHLHYGVPSRFSGSGSGTDFTLTISSLQPEDFATYYCQQYS |
| | GLPYTFGQGTKVEIK |
| SEQ ID NO: 2 | VH VH6L-BM (disclosed in WO2012/163520) |
| | YYQLVESGGGLVQPGGSLRLSCAADGFLFSGYAMSWIRQAPGKGLE |
| | WIGQISGSGGSTVYNYNVLGRFTISRDNSKNTLYLQMNSLRAEDTA |
| | VYYCARDTGYFDHWGQGTLVTVSS |
| SEQ ID NO: 3 | VL 6HVL_1 |
| | DQMTQSPSSLSASVGDRVTITCNAWWNISNYLAWYQQKPGKAPKLL |
| | IFDAKYRAPEVPSRFSGSGSGSQFTLTISSLQPEDFATYYCQQYSG |
| | WPFTFGQGTKVEIK |
| SEQ ID NO: 4 | VH 6HVL_1 |
| | GQLVESGGGLVKPGGSLRLSCKASGFYWGPGAMSWVRQAPGKGLEW |
| | VGSISPKGGSEWFNPEIKGRFTISRDNNQDTLYLQMNSLRAEDTAV |
| | YYCARDIGFFDTWGQGTLVTVSS |
| SEQ ID NO: 5 | VL 6HVL_2 |
| | DQMTQSPSSLSASVGDRVTITCKALWEVRDYLAWYQQKPGKAPKLL |
| | IFDGEYRAPEVPSRFSGSGSGTEFTLTISSLQPEDFATYYCQHYAG |
| | WPYTFGQGTKLEIK |
| SEQ ID NO: 6 | VH 6HVL_2 |
| | GQLVESGGGLVQPGGSLRLSCAASGFYWGPGQMSWVRQAPGKGLEW |
| | VGSIDTKGDDEWYDPKFDGRFTISRDNAKNSLYLQMNSLRAEDTAV |
| | YYCARDIGFFDTWGQGTLVTVSS |
| SEQ ID NO: 7 | VL 6HVL_3 |
| | IQMTQSPSSLSASVGDRVTITCRALWEIRDYLAWYQQKPGKAPKLL |
| | IFDGEYRAPEVPSRFSGSGSGTDFTLTISSLQPEDFATYYCQHYAG |
| | WPYTFGQGTKLEIK |
| SEQ ID NO: 8 | VH 6HVL_3 |
| | GQLVESGGGLVQPGGSLRLSCAASGFYWGPGQMSWVRQAPGKGLEW |
| | VGSIDTKGRDEWYDPKFDGRFTISRDNSKNTLYLQMNSLRAEDTAV |
| | YYCARDIGFFDTWGRGTLVTVSS |
| SEQ ID NO: 9 | VL VH6L_1 |
| | AIQMTQSPSSLSASVGDRVTITCHGSYWLSSEVAWYQQKPGKAPKL |
| | LIYDGDDVVPEVPSRFSGSGSHEDYTLTISSLQPEDFATYYCQQYR |
| | YHPYTFGHGTKVEIK |
| SEQ ID NO: 10 | VH VH6L_1 |
| | YIQLVESGGGLVKPGGSLRLSCAAGYEFTHQDMSWVRQAPGKGLEW |
| | VGSISPKGDHKYLNTKFIGRFTISRDNSKNTLYLQMNSLRAEDTAV |
| | YYCARDIGFFDTWGQGTLVTVSS |
| SEQ ID NO: 11 | VL VH6L_2 |
| | AIQMTQSPSSLSASVGDRVTITCHGSYWLSSEVAWYQQKPGKAPKL |
| | LIYDASSNYPSVPSRFSGSGSHEDYTLTISSLQPEDFATYYCQQYR |
| | YHPYTFGHGTKVEIK |
| SEQ ID NO: 12 | VH VH6L_2 |
| | YIQLVESGGGLVKPGGSLRLSCAAGYEFTHQDMSWVRQAPGKGLEW |
| | VGSISPKGDHKYLNTKFIGRFTISRDNSKNTLYLQMNSLRAEDTAV |
| | YYCARDIDFFDTWGQGTLVTVSS |
| SEQ ID NO: 13 | VL VH6L_3 |
| | AIQMTQSPSSLSASVGDRVTITCHGSYWLNSEVAWYQQKPGKAPKL |
| | LIYDGDTVYPEVPSRFSGSGSHEDYTLTISSLQPEDFATYYCQQYR |
| | YHPYTFGQGTKVEIK |
| SEQ ID NO: 14 | VH VH6L_3 |
| | YPQLVESGGGLVKPGGSLRLSCAASVLFKHQDMSWVRQAPGKGLEW |
| | VGSISPKGDHKYLNTKFIGRFTISRDNSKNTLYLQMNSLRAEDTAV |
| | YYCARDIDFFDEWGQGTLVTVSS |
| SEQ ID NO: 15 | LCDR-1 6HVL_4 |
| | QASYWLNSELA |
| SEQ ID NO: 16 | LCDR-2 6HVL_4 |
| | DGDDRYP |
| SEQ ID NO: 17 | LCDR-3 6HVL_4 |
| | QQYRYTPYT |
| SEQ ID NO: 18 | HCDR-1 6HVL_4 |
| | QDMS |
| SEQ ID NO: 19 | HCDR-2 6HVL_4 |
| | SIDPRGDHKYYNTKFID |
| SEQ ID NO: 20 | HCDR-3 6HVL_4 |
| | DLDFMDE |
| SEQ ID NO: 21 | VL 6HVL_4 |
| | AIYMHQEPSSLSASVGDRVTITCQASYWLNSELAWYQQKPGKAPKL |
| | LIYDGDDRYPEVPSRFSGSGSHEDYTLTISSLQPEDFATYYCQQYR |
| | YTPYTFGQGTKLEIK |
| SEQ ID NO: 22 | VH 6HVL_4 |
| | YPQLVESGGGLVQPGGSLRLSCEASVLFKHQDMSWVRQAPGKGLEW |
| | VGSIDPRGDHKYYNTKFIDRFTISRDNSKNTLYLQMNSLRAEDTAV |
| | YYCARDLDFMDEWGQGTLVTVSS |
| SEQ ID NO: 23 | LC 6HVL_4 |
| | AIYMHQEPSSLSASVGDRVTITCQASYWLNSELAWYQQKPGKAPKL |
| | LIYDGDDRYPEVPSRFSGSGSHEDYTLTISSLQPEDFATYYCQQYR |
| | YTPYTFGQGTKLEIKRTVAAPSVFIFPPSDEQLKSGTASVVCLLNN |
| | FYPREAKVQWKVDNALQSGNSQESVTEQDSKDSTYSLSSTLTLSKA |
| | DYEKHKVYACEVTHQGLSSPVTKSFNRGEC |
| SEQ ID NO: 24 | HC 6HVL_4 |
| | YPQLVESGGGLVQPGGSLRLSCEASVLFKHQDMSWVRQAPGKGLEW |
| | VGSIDPRGDHKYYNTKFIDRFTISRDNSKNTLYLQMNSLRAEDTAV |
| | YYCARDLDFMDEWGQGTLVTVSSASTKGPSVFPLAPSSKSTSGGTA |
| | ALGCLVKDYFPEPVTVSWNSGALTSGVHTFPAVLQSSGLYSLSSVV |
| | TVPSSSLGTQTYICNVNHKPSNTKVDKKVEPKSCDKTHT |
| SEQ ID NO: 25 | VL 6HVL_4 YHE |
| | AIQMTQSPSSLSASVGDRVTITCQASYWLNSELAWYQQKPGKAPKL |
| | LIYDGDDRYPEVPSRFSGSGSHEDYTLTISSLQPEDFATYYCQQYR |
| | YTPYTFGQGTKLEIK |
| SEQ ID NO: 26 | VH 6HVL_4 YHE |
| | YPQLVESGGGLVQPGGSLRLSCEASVLFKHQDMSWVRQAPGKGLEW |
| | VGSIDPRGDHKYYNTKFIDRFTISRDNSKNTLYLQMNSLRAEDTAV |
| | YYCARDLDFMDEWGQGTLVTVSS |
| SEQ ID NO: 27 | human VEGFA-121 |
| | MNFLLSWVHWSLALLLYLHHAKWSQAAPMAEGGGONHHEVVKFMDV |
| | YQRSYCHPIETLVDIFQEYPDEIEYIFKPSCVPLMRCGGCCNDEGL |
| | ECVPTEESNITMQIMRIKPHQGQHIGEMSFLQHNKCECRPKKDRAR |
| | QEKCDKPRR |
| SEQ ID NO: 28 | human IL6 |
| | MNSFSTSAFGPVAFSLGLLLVLPAAFPAPVPPGEDSKDVAAPHRQP |
| | LTSSERIDKQIRYILDGISALRKETCNKSNMCESSKEALAENNLNL |
| | PKMAEKDGCFQSGFNEETCLVKIITGLLEFEVYLEYLQNRFESSEE |
| | QARAVQMSTKVLIQFLQKKAKNLDAITTPDPTTNASLLTKLQAQNQ |
| | WLQDMTTHLILRSFKEFLQSSLRALRQM |
| SEQ ID NO: 29 | CL |
| | RTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQ |
| | GLSSPVTKSENRGEC |
| SEQ ID NO: 30 | CH1 |
| | ASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNT |
| | KVDKKVEPKSCDKTHT |
| SEQ ID NO: 31 | Fab 0182 VL |
| | TPVQMTQSPSSLSASVGDRVTITCHGFWDIRDYLAWYQQKPGKAPK |
| | LLIFDAKYRAPEVPSRFSGSGSGTEFTLTISSLQPEDFATYYCQQY |
| | SGWPFTFGQGTKVEIK |
| SEQ ID NO: 32 | Fab 0182 VH |
| | GQLVESGGGLVKPGGSLRLSCKASGFYWGPGAMSWVRQAPGKGLEW |
| | VGSIDLKGEDAWFDHEFSGRFTISRDNSKDTLYLQMNSLRAEDTAV |
| | YYCARDIGFFDTWGQGTLVTVSS |
| SEQ ID NO: 33 | Fab 0182 LC |
| | TPVQMTQSPSSLSASVGDRVTITCHGFWDIRDYLAWYQQKPGKAPK |
| | LLIFDAKYRAPEVPSRFSGSGSGTEFTLTISSLQPEDFATYYCQQY |
| | SGWPFTFGQGTKVEIKRTVAAPSVFIFPPSDEQLKSGTASVVCLLN |
| | NFYPREAKVQWKVDNALQSGNSQESVTEQDSKDSTYSLSSTLTLSK |
| | ADYEKHKVYACEVTHQGLSSPVTKSENRGEC |
| SEQ ID NO: 34 | Fab 0182 HC |
| | GQLVESGGGLVKPGGSLRLSCKASGFYWGPGAMSWVRQAPGKGLEW |
| | VGSIDLKGEDAWFDHEFSGRFTISRDNSKDTLYLQMNSLRAEDTAV |
| | YYCARDIGFFDTWGQGTLVTVSSASTKGPSVFPLAPSSKSTSGGTA |
| | ALGCLVKDYFPEPVTVSWNSGALTSGVHTFPAVLQSSGLYSLSSVV |
| | TVPSSSLGTQTYICNVNHKPSNTKVDKKVEPKSCDKTHT |
| SEQ ID NO: 35 | 6HVL4.1 VL |
| | AIQMTQSPSSLSASVGDRVTITCHGSYWLNSEVAWYQQKPGKAPKL |
| | LIYDASSNYPSVPSRFSGSGSHEDYTLTISSLQPEDFATYYCQQYR |
| | YHPYTFGQGTKVEIK |
| SEQ ID NO: 36 | 6HVL4.1 VH |
| | YIQLVESGGGLVKPGGSLRLSCAAGYEFTHQDMSWVRQAPGKGLEW |
| | VGSISPKGDHKYLNTKFIGRFTISRDNSKNTLYLQMNSLRAEDTAV |
| | YYCARDIDFFDTWGQGTLVTVSS |
| SEQ ID NO: 37 | 6HVL4.1 LC |
| | MTMITPSFGAFFLEIFNMKQSTIALALLPLLFTPVSKAAIQMTQSP |
| | SSLSASVGDRVTITCHGSYWLNSEVAWYQQKPGKAPKLLIYDASSN |
| | YPSVPSRFSGSGSHEDYTLTISSLQPEDFATYYCQQYRYHPYTFGQ |
| | GTKVEIKRTVAAPSVFIFPPSDEQLKSGTASVVCLLNNFYPREAKV |
| | QWKVDNALQSGNSQESVTEQDSKDSTYSLSSTLTLSKADYEKHKVY |
| | ACEVTHQGLSSPVTKSENRGE |
| SEQ ID NO: 38 | 6HVL4.1 HC |
| | YIQLVESGGGLVKPGGSLRLSCAAGYEFTHQDMSWVRQAPGKGLEW |
| | VGSISPKGDHKYLNTKFIGRFTISRDNSKNTLYLQMNSLRAEDTAV |
| | YYCARDIDFFDTWGQGTLVTVSSASTKGPSVFPLAPSSKSTSGGTA |
| | ALGCLVKDYFPEPVTVSWNSGALTSGVHTFPAVLQSSGLYSLSSVV |
| | TVPSSSLGTQTYICNVNHKPSNTKVDKKVEPKS |
| SEQ ID NO: 39 | VL 6HVL_5 |
| | AIYMHQEPSSLSASVGDRVTITCQASYWLNSELAWYQQKPGKAPKL |
| | LIYDGDTNYPEVPSRFSGSGSHEDYTLTISSLQPEDFATYYCQQYR |
| | YTPYTFGQGTKLEIK |
| SEQ ID NO: 40 | VH 6HVL_5 |
| | YPQLVESGGGLVQPGGSLRLSCEASVLFKHQDMSWVRQAPGKGLEW |
| | VGSIDPRGDHKYYNTKFIDRFTISRDNSKNTLYLQMNSLRAEDTAV |
| | YYCARDLDFMDEWGQGTLVTVSS |
| SEQ ID NO: 41 | VL 6HVL_6 |
| | AIYMHQEPSSLSASVGDRVTITCQASYWLNSELAWYQQKPGKAPKL |
| | LIYDGDDTYPEVPSRFSGSGSHEDYTLTISSLQPEDFATYYCQQYR |
| | YTPYTFGQGTKLEIK |
| SEQ ID NO: 42 | VH 6HVL_6 |
| | YPQLVESGGGLVQPGGSLRLSCEASVLFKHQDMSWVRQAPGKGLEW |
| | VGSISPRGDHKYYNTKFIDRFTISRDNSKNTLYLQMNSLRAEDTAV |
| | YYCARDLDFMDEWGQGTLVTVSS |
| SEQ ID NO: 43 | VL VH6L_4 |
| | IQMTQSPSSLSASVGDRVTITCRALWEIRDYLAWYQQKPGKAPKLL |
| | IFDGEYRAPEVPSRFSGSGSGTEFTLTISSLQPEDFATYYCQHYAG |
| | WPYTFGQGTKLEIK |
| SEQ ID NO: 44 | VH VH6L_4 |
| | GQLVESGGGLVQPGGSLRLSCAASGFYWGPGQMSWVRQAPGKGLEW |
| | VGSIDTKGDDEWYDPKFDGRFTISRDNSKNTLYLQMNSLRAEDTAV |
| | YYCARDIGFFDTWGRGTLVTVSS |
| SEQ ID NO: 45 | VL VH6L_5 |
| | AIQMTQSPSSLSASVGDRVTITCRALWEIRDYLAWYQQKPGKAPKL |
| | LIFDGEYRAPEVPSRFSGSGSGTDFTLTISSLQPEDFATYYCQHYA |
| | GWPYTFGQGTKLEIK |
| SEQ ID NO: 46 | VH VH6L_5 |
| | GQLVESGGGLVQPGGSLRLSCAASGFYWGPGQMSWVRQAPGKGLEW |
| | VGSIDTKGRDEWYDPKFDGRFTISRDNSKNTLYLQMNSLRAEDTAV |
| | YYCARDIGFFDTWGRGTLVTVSS |
| SEQ ID NO: 47 | LC 6HDL2.05 |
| | DIQMTQSPSSLSASVGDRVTITCRASQGISNYLAWYQQKPGKAPKL |
| | LIYDGDDRYPEVPSRFSGSGSGTDFTLTISSLQPEDFATYYCQQYY |
| | STPYTFGQGTKLEIKRTVAAPSVFIFPPSDEQLKSGTASVVCLLNN |
| | FYPREAKVQWKVDNALQSGNSQESVTEQDSKDSTYSLSSTLTLSKA |
| | DYEKHKVYACEVTHQGLSSPVTKSENRGEC |
| SEQ ID NO: 48 | HC 6HDL2.05 |
| | YPQLVESGGGLVQPGGSLRLSCEASVLFKHQDMSWVRQAPGKGLEW |
| | VGSISPSGGSTYYNDNVLGRFTISRDNSKNTLYLQMNSLRAEDTAV |
| | YYCARDLDFMDEWGQGTLVTVSSASTKGPSVFPLAPSSKSTSGGTA |
| | ALGCLVKDYFPEPVTVSWNSGALTSGVHTFPAVLQSSGLYSLSSVV |
| | TVPSSSLGTQTYICNVNHKPSNTKVDKKVEPKSCDKTHTSPPGSGM |
| | ASMTGGQQMG |

### EXAMPLES

The following examples are provided to aid the understanding of the present invention, the true scope of which is set forth in the appended claims. It is understood that modifications can be made in the procedures set forth without departing from the spirit of the invention.

### Example 1

### Generation of Bispecific Anti-VEGF/Anti-6 Fab Fragment

A bispecific anti-VEGF/anti-IL-6 Fab fragment was generated by providing antibodies having separate, non-overlapping paratopes that bind to VEGF and IL-6 using a method similar to what has been described before, e.g. in WO2012/163520.

Here, two distinct phage display libraries of synthetic Fab fragments were utilized, wherein in the first phage display library residues within the CDR-H1, CDR-H3 and CDR-L2 regions of the Fab fragments were diversified, and wherein in the second phage display library residues within the CDR-L1, CDR-L3 and CDR-H2 regions of the Fab fragments were diversified. In each library the respective other three CDR regions were kept non-diversified and -contrary to the method of WO2012/163520 which used invariant non-binding, germline-like (“dummy”) sequences-represented a paratope capable of binding to VEGF-A.

In the case of the first library, the paratope capable of binding to VEGF-A was derived from the VEGF-A binding paratope described in WO 2021/198034.

In the case of the second library, the VEGF-A binding paratope was obtained as follows:

For naïve selection, phage library panning was performed with a library where CDR-H1, CDR-H3 and CDR-L2 had been diversified as described in WO2012/163520. The remaining CDR sequences were kept constant using a non-binding, germline-like sequence. In 4 rounds, wherein the first round was performed with 100 nM of biotinylated VEGF-121 or VEGF-165 pre-immobilized on Dynabeads M-280 Streptavidin (Thermofisher catalog number 11206D). Panning rounds 2-4 were performed with 75, 15 and 3 nM of biotinylated target in solution respectively, followed by capture of Fab-on-phage/target complexes on Dynabeads M-280 Streptavidin. Phage/target/bead complex were washed multiple times with PBST and PBS buffer. Captured phage clones bearing target-specific Fabs were eluted from the M-280 beads using 100 mM DTT, used for infection of log-phase TG1 E. coli cells, and rescued using M13 K07 helper phage, according to standard protocols.

For screening of selection outputs, a polyclonal plasmid miniprep of the respective selection round was prepared from the infected TG1 E. coli cells. Plasmids were reformatted to produce soluble Fab in E. coli supernatants with a T7 tag at the C-terminus of the Fab CH1 domain. The ligated polyclonal plasmids encoding T7-tagged Fabs were transformed into TG1 E. coli cells (Zymo Research catalog number T3017), and single colonies were picked into microtiter plates. Soluble Fabs were expressed in microtiter plates and supernatants were clarified by centrifugation. Target binding was assessed by ELISA measurements against VEGF, as well as competition ELISA against VEGF receptor 2. Candidate binders were selected based on high binding signal to VEGF as well as good inhibition of receptor binding.

Binders were subsequently expressed and purified in larger volume and binding to VEGF was assessed using SPR measurements. One of the clones obtained was further optimized by an iterative protein engineering and testing strategy and integrated in the phage display library as an invariant sequence for CDR-H1, CDR-H3 and CDR-L2. In short, the protein engineering workstream consisted of initial rounds of scouting mutations to identify relevant beneficial mutations, followed by two sequential rounds of affinity maturation based on oligonucleotide-based generation of mutant libraries and phage-display based selection followed by screening and further testing.

In both libraries, the CH1 domain of the Fab fragments was fused via a linker to a truncated gene-III protein to facilitate phage display. Thus, one library was intended to screen for bispecific Fab fragments wherein the IL6 paratope comprises amino acid residues from the CDR-H1, CDR-H3 and CDR-L2 (herein termed the “6HVL” library) and the other library was intended to screen for bispecific Fab fragments wherein the IL6 paratope comprises amino acid residues from CDR-H2, CDR-LI and CDR-L3 (herein termed the “VH6L” library).

Each library was enriched for binders against human IL-6 by phage library panning. Following panning, plasmid minipreps were generated for both enriched pools of phagemid vectors. The minipreps were digested with a restriction enzyme to excise the region encoding the truncated gene-III protein and re-circularized by ligation to obtain pools of expression vectors encoding soluble Fab fragments that were enriched for IL-6 binders. These vector pools were transformed into TG1 E. coli cells and individual colonies were picked and cultured for soluble expression of individual Fab clones in microtiter plates. The supernatants comprising soluble Fab fragments were screened for binding to IL-6 and VEGF-A using standard ELISA methods.

Based on the screening data, bispecific anti-VEGF/anti-IL-6 Fab fragments were selected, and TG1 clones producing specific binders were subjected to DNA plasmid preparation and sequencing, to obtain pairs of VH and VL sequences that together encode one bispecific Fab fragment specifically binding both to IL-6 and to VEGF-A from each library, respectively:

The clones were 6HVL_1, characterized by a heavy chain of SEQ ID NO:03 and a light chain of SEQ ID NO:04, and VH6L_1, characterized by a heavy chain of SEQ ID NO: 09 and a light chain of SEQ ID NO:10.

### Example 2

### Expression and Characterization of Bispecific Anti-VEGF/Anti-IL-6 Fab Fragments 6HVL_1 and VH6L_1

The resulting bispecific anti-VEGF/anti-IL-6 Fab fragments were characterized. The vectors obtained as described under Example 1 were transformed into TG1 E. coli cells, and for both 6HVL_1 and VH6L_1 an individual colony was cultured for soluble expression of the bispecific antibody Fab fragment. The bispecific antibody was purified from the TG1 culture supernatant by affinity chromatography. Binding to IL-6 from human and cynomolgus monkey IL6, human VEGF121 and human VEGF165 of the bispecific antibodies 6HVL_1 and VH6L_1 were assessed.

### Example 3

### Characterization of Bispecific Anti-VEGF/Anti-IL-6 Fab Fragments 6HVL_1 and VH6L_1

### IL-6 Binding Kinetics as Assessed by Surface Plasmon Resonance (SPR);

Surface plasmon resonance (SPR) was used to measure binding kinetics and affinity of the representative VEGF-IL-6 Fab fragments to human and cynomolgus monkey IL-6 disclosed herein.

SPR analysis of the binding of the respective Fab fragments IL-6 from human and cynomolgus monkey was performed at 25° C. on a Biacore 8K instrument (Cytiva) using HBS-EP+ (1×; BR100669; Cytiva) as running buffer. A Human Fab Binder (28958325, Cytiva) was diluted at a final concentration of 10 μg/ml in 10 mM sodium acetate buffer, pH 5.0 and immobilized on a CM5 sensor chip using standard amine coupling chemistry. This immobilization procedure resulted in a ligand density of about 5000 resonance units (RU). The reference channel was treated accordingly.

Prior to the protein measurements five startup cycles were performed for conditioning purposes. On each cycle, HBS-EP+ buffer was flowed for 120 s followed by the regeneration of the derivatized chip surface by applying 10 mM Glycine buffer pH2.0 for 60 s. Fab fragment at a concentration of 75 nM was captured on this surface for 60 s at a flow rate of 10 μl/min in HBS-EP+ buffer. No Fab fragment was applied to the reference channel. Subsequently, human or cynomolgus monkey IL-6 were applied in an appropriate dilution series in HBS-EP+ buffer at a flow rate of 30 μl/min (contact time 180 s, dissociation time 720 s). Regeneration of the derivatized chip surface was achieved as described above. Data were evaluated with 8K Evaluation software (Biacore Insight Evaluation 3.0). Double referencing was used and the 1:1 Binding model was used to fit the raw data.

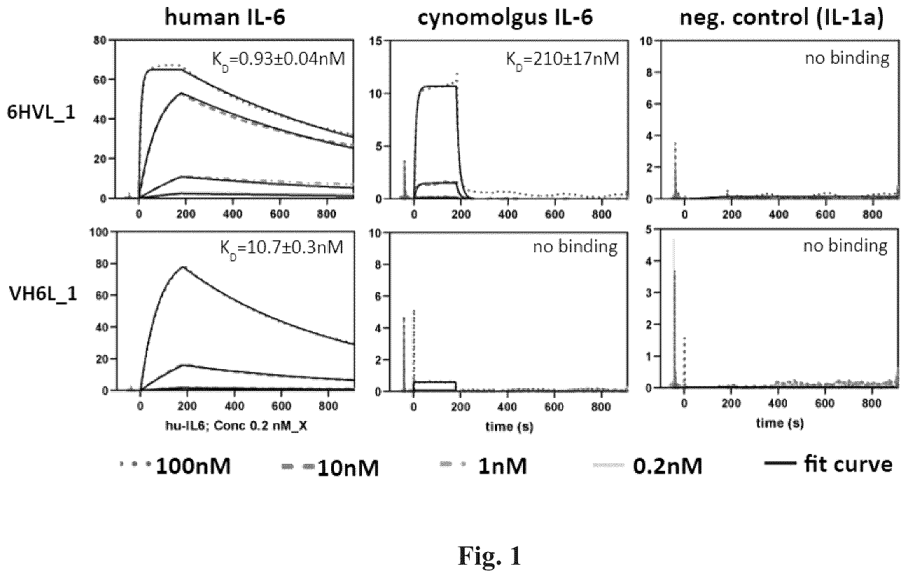

FIG. 1 shows representative SPR traces and fit curves determined for the Fab fragments tested, with the corresponding Fab names provided in the graphs. The data is depicted for the binding to human and cynomolgus monkey IL-6, and to IL-1 alpha (IL-la) as a negative control. The affinities provided in the graphs correspond to mean and standard deviation for three independent experiments.

We observe clear binding of 6HVL_1 and VH6L_1 to human IL-6. Only VH6L_1 shows a significant affinity towards cyIL-6, albeit with an apparently very fast off-rate. No binding to the negative control target IL-la is observed. The results of fitting the SPR data is shown in Table 1. Data were averaged for three experiments, and the standard deviation is provided for the dissociation constant KD. For 6HVL_1, we observe an affinity of KD=0.9 nM, while for VH6L_1, the affinity is KD=10.7 nM.

TABLE 1
Affinity of indicated antibodies to human and cynomolgus IL6

| | human IL6 | | cynomolgus IL6 | |
|---|---|---|---|---|
| | K_{D} [nM] | SD [nM] | K_{D} [nM] | SD [nM] |
| 6HVL_1 | 0.93 | 0.04 | 210 | 17 |
| VH6L_1 | 10.7 | 0.3 | — | — |

### VEGF Binding as Assessed by Competition ELISA:

To test which antibody concentration is needed to block the interaction of VEGF121 and VEGF165 with their receptor, competition ELISA experiments with 6HVL_1 and VH6L_1 were performed. The VEGF-binding Fab fragment ranibizumab was used as the positive control, and an experiment using buffer only was used as the negative control. In short, a 1:3 dilution series of all samples-starting with 20 nM-were mixed with a constant concentration of 10 pM VEGF121 (Humanzyme HZ-1206) or 10 pM VEGF165 (Humanzyme HZ-1153) and incubated for 90 min. This mixture was then transferred to Maxisorp-plates that had been coated with the VEGF-receptor 1 (VEGF-R1, R&D systems, 1 μg/ml in NaHCO₃, pH 9.4) after blocking the Maxisorp plate surface with 2% MPBST. The contact time between the Fab-Antigen mixture and the receptor-coated plate was restricted to 10 min at RT in order to minimize interference with the binding equilibria. Following incubation and 2 washing steps the detection of VEGF121/VEGF165 on the VEGFR1-coated plate was performed using biotinylated anti-VEGF mAb (BAF203, R&D systems) and horseradish-peroxidase-labeled streptavidin (HRP-streptavidin). The latter is detected exploiting the chromogenic conversion of the HRP substrate 3,3′,5,5′-tetramethylbenzidine TMB to 3,3′,5,5′-tetramethylbenzidine diamine, which can be followed by a change in absorbance at 450 nm. TMB was prewarmed to room temperature and incubated on the plate for 5 min before being quenched by the addition of IN H₂SO₄.

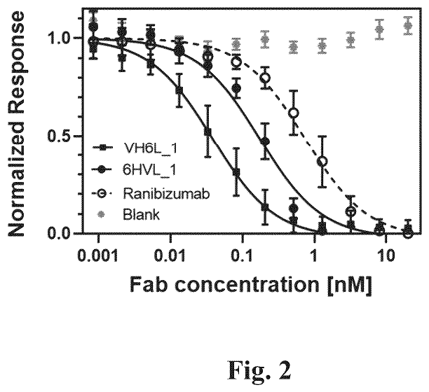

Results with the target VEGF165 are shown in FIG. 2 and in Table 5 and Table 6. Clearly, both VH6L_1 and 6HVL_1 display a much improved ability to compete with the binding of both VEGF165 and VEGF121 to VEGFR1 compared to Ranibizumab, a clinically well established VEGF-A antagonist.

### Example 4

### Improvement of Bispecific Anti-VEGF-A/Anti-IL6 Fab Fragments

As illustrated above, both antibodies exhibited either no or low crossreactivity with cynomolgus IL6, which, however, is desired for clinical development. In addition to that, the treatment of ocular vascular diseases requires injection of the therapeutic into the eye, and consequently an optimal therapeutic should display high affinity for the target antigen and a high concentration to maximize durability of the therapeutic effect and patient convenience. For the intended purpose, it is therefore desired to further improve the molecules that were initially identified.

Several rounds of maturations were performed by introducing distinct amino acid substitutions in the VH and VL domain. During the maturations candidate antibodies derived from both “parental” antibodies 6HVL_1 and VH6L_1 were screened and selected based on their desired properties with respect to yield, affinity, simultaneous antigen binding, hydrophilicity, stability, viscosity and other parameters.

Improved candidate antibodies 6HVL_2, 6HVL_3 and 6HVL_4 and VH6L_2 and VH6L_3 were selected from a plurality of tested candidate antibody molecules from each round of maturations. Candidate selection was based on the desired properties, particularly improving human IL6 binding and cynomolgus monkey IL6 crossreactivity, while assuring syringeability at high concentrations and maintaining other advantageous characteristics, e.g. VEGF-A affinity and thermal stability.

Improved candidate antibody 6HVL_4 was selected as a preferred candidate from a plurality of tested candidate antibody molecules.

TABLE 2
Amino acid sequences of indicated bispecific Fab fragments
(the numbers refer to the SEQ ID NOs as used herein)

| | VL | VH | light chain | heavy chain |
|---|---|---|---|---|
| 6HVL_2 | 5 | 6 | | |
| 6HVL_3 | 7 | 8 | | |
| VH6L_2 | 11 | 12 | | |
| VH6L_3 | 13 | 14 | | |
| 6HVL_4 | 21 | 22 | 23 | 24 |
| 6HVL_4_YHE | 25 | 26 | | |

All Fab fragments included the same constant regions as comprised in the full length light chain and heavy chain amino acid sequences for antibody VH6L_4, i.e. a CL having SEQ ID NO:29 and a CH1 having SEQ ID NO:30.

The candidate antibodies were expressed as described in Example 2.

### Example 5

### Antigen Binding Kinetics of Improved Anti-VEGF-A/Anti-IL6 Fab Fragments

Binding kinetics to human and cynomolgus monkey IL6 and the competition IC50 for VEGF/VEGFR1 competition for the candidate antibodies were assessed as described above using the indicated Fab fragments (amino acid sequence as illustrated in Table 2 and Example 2). In order to determine the potency of the antibodies of the invention to prior art, the following controls were employed: the bispecific antibody VH6L (VH/VL sequences disclosed in WO2012/163520, herein termed “VH6L-BM”), the anti-VEGF antibody Ranibizumab (INN) and an anti-IL6 antibody cross-reactive between human and cynomolgus IL6 as disclosed in WO2014/074905 (positive control). The aforementioned prior art antibodies were prepared by recombinant expression.

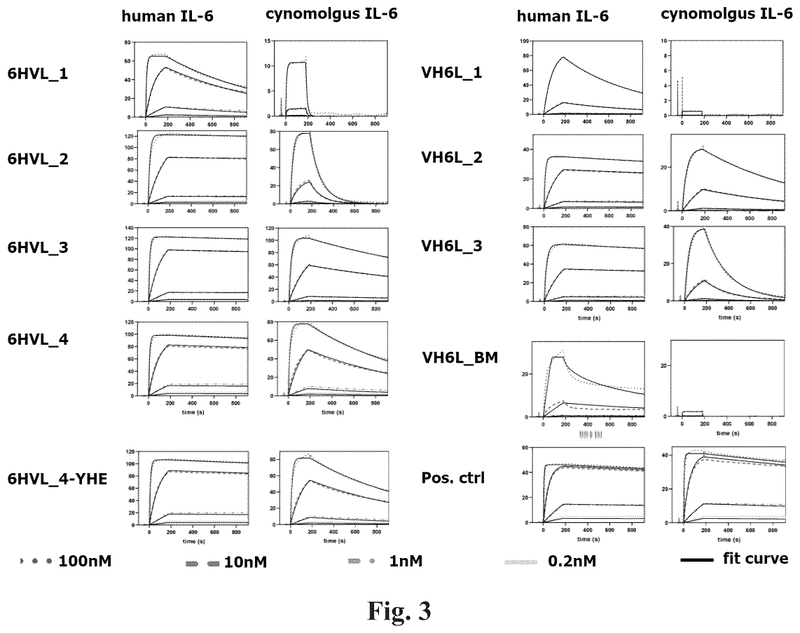

FIG. 3 and Tables 3 and 4 show the results of the assessment of human and cynomolgus IL6 binding. 6HVL_4 and 6HVL_4-YHE, a variant of 6HVL_4 having three further framework amino acid mutations, exhibit improved human IL6 binding over the initially selected parental molecule as well as cynomolgus IL6 cross-reactivity in a pharmacologically relevant range.

TABLE 3
SPR human IL6

| | K_{D} [nM] | SD [nM] |
|---|---|---|
| 6HVL_1 | 0.927 | 0.042 |
| 6HVL_2 | 0.036 | 0.003 |
| 6HVL_3 | 0.043 | 0.013 |
| 6HVL_4 | 0.069 | 0.002 |
| 6HVL_4_YHE | 0.065 | 0.002 |
| VH6L_1 | 10.7 | 0.3 |
| VH6L_2 | 0.157 | 0.003 |
| VH6L_3 | 0.191 | 0.027 |
| VH6L_BM | 3.5 | 0.6 |
| Pos. control | 0.035 | 0.007 |

TABLE 4
SPR cynomolgus IL6

| | K_{D} [nM] | SD [nM] |
|---|---|---|
| 6HVL_1 | 210 | 16.57 |
| 6HVL_2 | 23.37 | 0.84 |
| 6HVL_3 | 0.98 | 0.07 |
| 6HVL_4 | 1.48 | 0.09 |
| 6HVL_4_YHE | 1.39 | 0.04 |
| VH6L_1 | n.s.* | |
| VH6L_2 | 1.3E−03 | 0.29 |
| VH6L_3 | 5.3E−03 | 0.51 |
| VH6L_BM | n.s.* | |
| Pos. control | 1.8E−04 | 0 |

*no evaluable signal

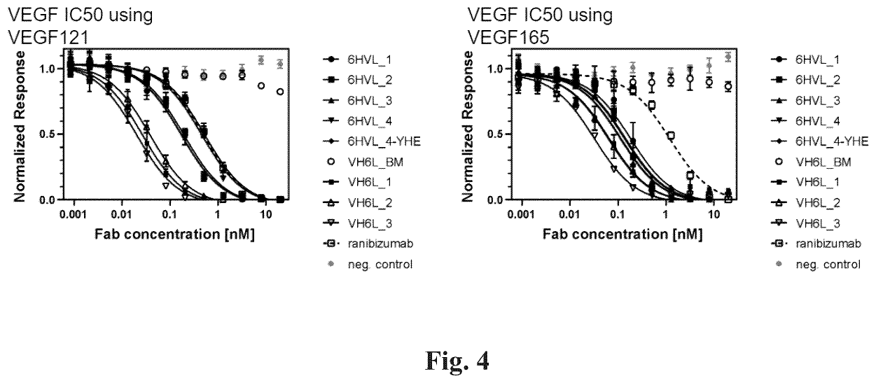

FIG. 4 and Tables 5 and 6 show the results of the assessment of VEGF Binding 5 as assessed by competition ELISA using human VEGF121 and human VEGF165. FIG. 4 illustrates that 6HVL_BM, the prior art molecule, exhibits an affinity that is too low to be detected under the conditions of the assay and therefore definitely much lower than the affinity of the antibodies of the invention.

TABLE 5
IC50 VEGF121

| | IC50 [pM] | SEM [nM] |
|---|---|---|
| 6HVL_1 | 173 | 12 |
| 6HVL_2 | 194 | 13 |
| 6HVL_3 | 171 | 11 |
| 6HVL_4 | 458 | 30 |
| 6HVL_4_YHE | 528 | 35 |
| VH6L_1 | 26 | 2 |
| VH6L_2 | 38 | 3 |
| VH6L_3 | 20 | 1 |
| Ranibizumab | 503 | 25 |

TABLE 6
IC50 VEGF165

| | IC50 [pM] | SEM [nM] |
|---|---|---|
| 6HVL_1 | 182 | 17 |
| 6HVL_2 | 143 | 13 |
| 6HVL_3 | 114 | 11 |
| 6HVL_4 | 111 | 10 |
| 6HVL_4_YHE | 135 | 13 |
| VH6L_1 | 65 | 6 |
| VH6L_2 | 62 | 6 |
| VH6L_3 | 32 | 3 |
| Ranibizumab | 1184 | 81 |

### Example 6

### Simultaneous Binding of Anti-VEGF/Anti-IL-6 Fab Fragments

Simultaneous binding to the antibodies of the invention to their targets was assessed as follows by surface plasmon resonance using an immobilized anti-Fab antibody to capture the anti-VEGF/anti-IL-6 Fab fragment of the invention:

Approximately 5000 resonance units (RU) of an anti-Fab antibody (Cytiva 28958325) were immobilized to a Series S Sensor Chip CM5 (Cytiva BR100530) using standard amine coupling chemistry. As running and dilution buffer, HBS-P+ (10 mM HEPES, 150 mM NaCl pH 7.4, 0.05% Surfactant P20) was used and the temperature of the flow cell was set to 25° C.

The anti-VEGF/anti-IL-6 Fab fragment was captured via Kappa chain by injecting a 10 μg/mL solution for 30 sec at a flow rate of 5 μL/min forming an anti-Fab antibody/anti-VEGF/anti-IL-6 Fab complex. Both antigens, human VEGFA121 (in-house production, PIAA1779-010) and human IL-6 (commercial, Peprotech #200-06) were added sequentially or simultaneously to allow the formation of a complex comprising anti-Fab antibody, anti-VEGF/anti-IL-6 Fab, human VEGFA and human IL-6. The corresponding SPR response unit curves (Biacore T200, Cytiva) were monitored. For sequential binding, human VEGFA with a concentration of 300 nM was injected for 180 sec followed by an additional injection of human IL-6 with a concentration of 300 nM for 180 sec. Same concentrations were also injected in reverse order (with first human IL-6 followed by human VEGFA). Similarly, a mixture of both antigens was injected for 180 s at a concentration of 300 nM each. After each experiment the surface was regenerated by injecting 10 mM Glycine pH 2.1 for 60 sec at a flow rate of 5 μL/min. Bulk refractive index differences were corrected by subtracting blank injections and by subtracting the response obtained from the control flow cell without captured Fab.

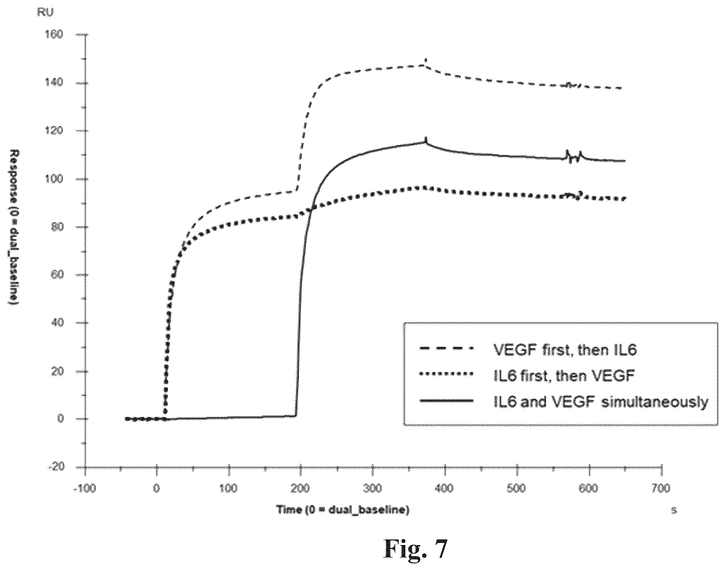

Results are shown in FIG. 7. Addition of human VEGF-A to the anti-Fab/anti-VEGF/anti-IL-6 Fab complex led to a binding and an anti-Fab/Fab/VEGF-A complex formation. Sequential addition of human IL-6 led to the formation of the anti-Fab/DutaFab/VEGF-A/IL-6 complex (dashed curve). This clearly demonstrated that simultaneous binding of human VEGF-A and human IL-6 to the anti-VEGF/anti-IL-6 Fab is possible.

In the reversed order, adding human IL-6 first followed by sequential addition of human VEGF-A, a reduced simultaneous binding was noticeable (dotted line). This indicated that binding of human IL-6 first is interfering sterically with the binding of human VEGF-A leading to a reduced binding ability between anti-VEGF/anti-IL-6 Fab and human VEGF-A but still is possible.

In presence of both targets, the human IL-6 binding seemed to be preferred and the binding to human VEGF-A was reduced (solid line). This effect was plausible due to the inherent higher affinity of the anti-VEGF/anti-IL-6 Fab to human IL-6 compared to human VEGF-A.

In another assay, blocking of VEGF-R2 by anti-VEGF/anti-IL-6 Fab fragments in presence of IL-6 were assessed by an inhibition assay with surface plasmon resonance using immobilized VEGF-A:

To show the simultaneous binding of human VEGF-A and human IL-6 to the anti-VEGF/anti-IL-6 Fab fragment, the human VEGF Receptor 2 (VEGFR2, commercial R&D Systems 357-KD) was immobilized to a Series S Sensor Chip CM5 (Cytiva BR100530) using standard amine coupling chemistry resulting in a surface density of approximately 11000 resonance units (RU). As running and dilution buffer, HBS-P+ (10 mM HEPES, 150 mM NaCl pH 7.4, 0.05% Surfactant P20) was used.

As reference, a 1:2 dilution series of 0-200 nM anti-VEGF/anti-IL-6 Fab fragment in a 50 nM human VEGFA solution was used and the VEGFA & VEGFR2 inhibition tested. The anti-VEGF/anti-IL-6 Fab fragment/VEGFA mixtures were injected onto the immobilized VEGFR2 surface for 30 sec at a flow rate of 5 μL/min. After a dissociation phase for 60 sec, the VEGFR2 surface was regenerated by injecting 5 mM NaOH for 30 sec at a flow rate of 5 μL/min. Bulk refractive index differences were corrected by subtracting blank injections and by subtracting the response obtained from the blank control flow cell. For evaluation, the binding response five seconds after inject end was taken. The derived response in RU was transformed to a binding response relative to the initial signal corresponding to the ligand(s) without bispecific Fab. IC50 values were calculated using a 4-parameter logistic model (XLfit, ID Business Solutions Ltd.)

In addition to the reference, dilutions of 0-200 nM anti-VEGF/anti-IL-6 Fab fragment in solutions with having 10 nM human IL-6 present were pre-incubated for 15 minutes and tested to calculate the IC50 values (FIG. 3).

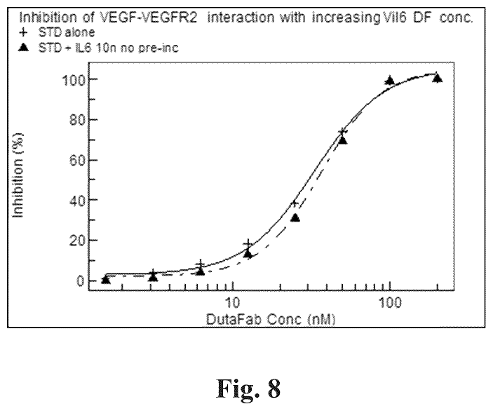

Results are shown in FIG. 8. The graph shows the inhibition of the VEGFR2/VEGF-A interaction dependent on the concentration of competing anti-VEGF/anti-IL-6 Fab. With no anti-VEGF/anti-IL-6 Fab present 100% VEGFR2/VEGF-A binding (0% inhibition) was achieved whereas increasing anti-VEGF/anti-IL-6 Fab concentrations increased the inhibition (solid line with crosses). The addition of human IL-6 mimicking therapeutic relevant conditions did not impact the degree of inhibition of VEGFR2/VEGF-A and resulted in very similar IC50 values (IC50=33 nM without human IL-6 (dashed line with triangles), IC50=37 nM with additional human IL-6 (dashed black line with triangles)).

In a third assay, the influence of VEGF-binding on IL6 activity was assessed as follows by a cell-based IL-6 specific reporter gene assay:

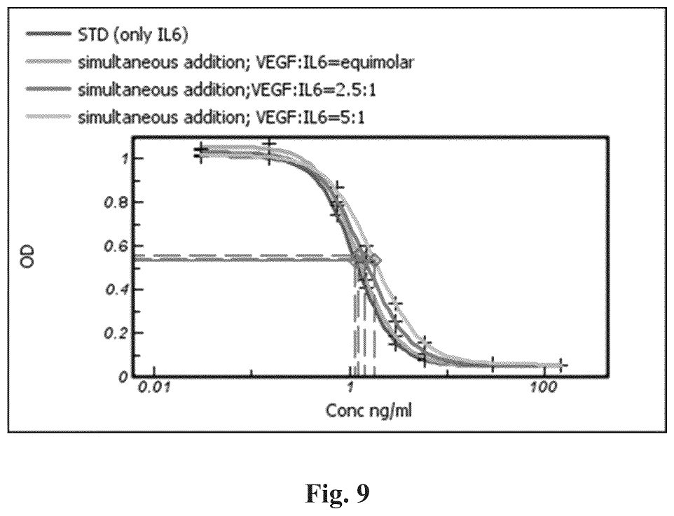

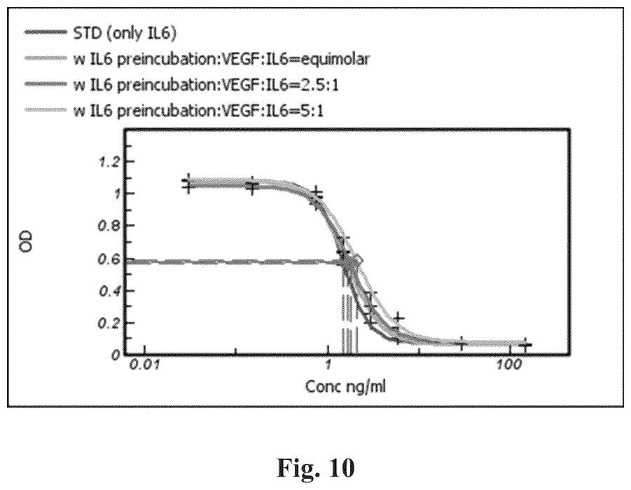

To assess the simultaneous binding of human VEGFA and human IL-6 to the anti-VEGF/anti-IL-6 Fab an IL-6 specific cell-based reporter gene assay with a reporter cell line, HEK-Blue™ IL-6 cells (InvivoGen), was used. The cells are incubated with the anti-VEGF/anti-IL-6 Fab and human IL-6 for 20 +/−1 hours in the absence and presence of human VEGF-A excess either by adding simultaneously (FIG. 9) or after preincubation of bispecific Fab and human IL-6 (FIG. 10). Binding of human IL-6 to its receptor IL-6R on the surface of HEK-Blue™ IL-6 cells triggers a signaling cascade through the tyrosine kinases of the Janus family (JAK1, JAK2 and Tyk2) leading to the activation of the signal transducer and transcription activator 3 (STAT3) and the subsequent secretion of SEAP (secreted embryonic alkaline phosphatase). In case of the binding of the anti-VEGF/anti-IL-6 Fab to human IL-6, the signaling is inhibited and SEAP is not produced. SEAP levels in cell culture supernatants are subsequently quantified by adding the QUANTI-Blue SEAP substrate to an aliquot of the supernatant. The SEAP converts the QUANTI-Blue substrate to a product that can be measured using a plate reader at 650 nm absorbance. The simultaneous binding of human VEGFA & human IL-6 is then assessed by graphing the mean absorbance versus the concentration of the anti-VEGF/anti-IL-6 Fab and the data are fitted to a constrained 4-parameter curve. The relative potency (inhibitory concentration) of a sample is calculated using the 4-parameter logistic curve fit.

Results are shown in FIGS. 9 and 10.

FIG. 9 shows the results without preincubation. The titration of increasing amounts of anti-VEGF/anti-IL-6 Fab showed a clear dose-response curve with a calculated IC₅₀ value of 1.134 ng/mL (˜22.5 pM) showing a clear inhibition of human IL-6 reaction by increasing amounts of anti-VEGF/anti-IL-6 Fab. To address simultaneous binding of human IL-6 and VEGFA to the bispecific Fab, both target molecules were incubated simultaneously and the human IL-6 effect measured. Regardless of the chosen ratio human VEGFA: human IL-6 (1:1/2.5:1/5:1) only a slight reduction of effective IC₅₀ value was noticed. The value changed slightly from IC₅₀=1.134 ng/ml in the absence of human VEGFA to IC₅₀=1.724 ng/mL when human VEGF-A was present in 5fold excess, a situation which reflects closely the in vivo relevant conditions.

FIG. 10 shows the results with preincubation illustrating that binding of IL6 does not influence the IL6 binding.

### Example 7

Binding of anti-VEGF/anti-IL-6 Fab fragments to IL-6 as determined by x-ray crystallography and proposed mode of action

IL-6 signaling is initiated by formation of a hexameric complex of IL6 with its non-signaling co-receptor IL6R and the cytokine receptor gp130. Here, three epitopes (sites 1, 2 and 3) have been defined to identify the contact surfaces that are forming in the complex (Boulanger M J et al., Science 2003, 27; 300 (5628); 2101-4.). IL-6 first binds to IL-6R through the interaction surface termed as “site 1”. “Site 2” is the epitope formed by the binary complex of IL-6 and IL-6R, which interacts with domains 2 and 3 of gp130. The subsequent interaction between “site 3” of IL6 and the domain 1 of gp130 leads to formation of a dimer of the IL6/IL6R/gp130 trimer and hence to formation of the hexameric signaling complex.

To understand which epitopes of IL6 are bound by our two series of Fabs (6HVL and VH6L), we carried out a structural analysis of the complexes between IL-6 and antibody Fabs which are representative of the anti-VEGF/anti-IL-6 Fab fragments of the invention, respectively. The utilized Fab 6HVL4.1 is very closely related to Fab 6HVL_2 and differs by just two mutations, and Fab 0182 is most closely related to Fab VH6L_1. Due to the fact that all 6HVL clones are derived from the same Fab (6HVL_1), and likewise all VH6L clones are derived from the Fab VH6L_1, respectively, it can be safely assumed that the structural results obtained in the following are applicable across each of the individual series of Fabs. Formation of the complex of Fab and IL-6 and analysis of the complex structure was performed by x-ray crystallography as follows:

The IL6-Fab complexes were prepared by mixing equal molar amounts of the Fab fragment 0182 (light chain amino acid sequence SEQ ID NO: 33, heavy chain amino acid sequence SEQ ID NO: 34) or 6HVL4.1 (light chain amino acid sequence SEQ ID NO: 37, heavy chain amino acid sequence SEQ ID NO: 38), respectively, with IL-6 (PeproTech, Lot #031316-2).

After 90 minutes of incubation on ice, the protein complex was concentrated to 23.1 mg/ml for Fab fragment 0182 and 21.3 mg/ml for 6HVL4.1. Initial crystallization trials were performed in sitting drop vapor diffusion setups at 21° C.

For Fab fragment 0182, needle shaped crystals appeared within two days out of 0.1M MgCl₂, 0.1 M sodium citrate pH 5, 15% (w/v) PEG 4000. Crystals were subsequently used in seeding experiments and large tetragonal shaped crystals could be obtained out 0.1 M calcium acetate, 12% (w/v) PEG 8000, 0.1 M sodium cacodylate, pH 5.5.

For 6HVL4.1, rhombohedral shaped crystals appeared within one day out of 0.2 M ammonium sulfate, 0.1 M Tris, pH 7.5 pH and 20% (w/v) PEG MME 5000.

For data collection the collection crystals were flash cooled at 100K in crystallization solution supplemented with 15% ethylene glycol. X-ray diffraction data were collected at a wavelength of 0.9999 A for Fab fragment 0182 and of 0.9982 Å fir 6HVL4.1 using a PILATUS 6M detector at the beamline X10SA of the Swiss Light Source (Villigen, Switzerland). Data have been processed with XDS (Kabsch, W., XDS. Acta Cryst. D66, 125-132 (2010)), scaled with AIMLESS (P. R. Evans and G. N. Murshudov “How good are my data and what is the resolution?” Acta Cryst. (2013). D69, 1204-1214) and analysed for anisotropy with STARANISO (Tickle, I. J., Flensburg, C., Keller, P., Paciorek, W., Sharff, A., Vonrhein, C., Bricogne, G. (2018). STARANISO (http://staraniso.globalphasing.org/cgi-bin/staraniso.cgi). Cambridge, United Kingdom: Global Phasing Ltd.).

The crystals of the complex including Fab 0182 belong to space group P21 with cell axes of a=65.93 Å, b=65.46 Å, c=159.30λ with β=91.65° and diffract to a resolution of 2.18 Å.

The crystals of the complex including 6HVL4. 1 belong to space group P212121 with cell axes of a=57.56 Å, b=64.98 Å, c=203.68 Å and diffract to a resolution of 1.94 Å.

The structure was determined by molecular replacement with PHASER (McCoy, A. J., Grosse-Kunstleve, R. W., Adams, P. D., Winn, M. D., Storoni, L. C., & Read, R. J. Phaser crystallographic software. J Appl Cryst. 40, 658-674 (2007)) using the coordinates of an in house Fab and IL-6 (pdb entry lalu) as search model. Difference electron density was used to change amino acids according to the sequence differences. The structure was refined with programs from the CCP4 suite (Winn, M. D. et al. Overview of the CCP4 suite and current developments. Acta. Cryst. D67, 235-242 (2011)) and BUSTER (Bricogne, Blanc, G. E., Brandl, M., Flensburg, C., Keller, P., Paciorek, W., Roversi, P., Sharff, A., Smart, O. S., Vonrhein, C., Womack, T. O. Buster version 2.9.5 Cambridge, United Kingdom: Global Phasing Ltd. (2011)). Manual rebuilding was done with COOT (Emsley, P., Lohkamp, B., Scott, W. G., Cowtan, K. Features and Development of Coot. Acta Cryst. D66, 486-501 (2010)).

Data collection and refinement statistics are summarized in Table 7. All graphical presentations were prepared with PYMOL (The Pymol Molecular Graphics System, Version 1.7.4. Schrödinger, LLC.)

TABLE 7
Data collection and refinement statistics

| | |
|---|---|
| Fab0182-IL-6 | Fab 6HVL4.1-IL6 |
| complex | complex |

Data collection

| | | |
|---|---|---|
| Space group | P2₁ | P2₁2₁2₁ |

Cell dimensions

| | | |
|---|---|---|
| a, b, c (Å) | 65.930, 65.46, 159.30 | 57.56, 64.98, 203.68 |
| α, β, γ (°) | 90, 91.65, 90 | 90, 90, 90 |
| Resolution (Å) | 2.18 | 1.94 |
| Rₘₑₐₛ | 0.12 | 0.10 |
| I / σI | 9.98 (1.87) | 10.00 (0.85) |
| CC(1/2) | 0.996 (0.579) | 99.2 (98.6) |
| Completeness ellipsoidal | 48.1 | |
| overall (%) | | |
| Completeness ellipsoidal | | |
| Inner Shell (%) | | |
| Completeness ellipsoidal | 99.7 | |
| Outer Shell (%) | | |
| | 6.0 | |
| Redundancy | 3.80 (3.55) | 7.34 (7.55) |
| Refinement | | |
| Resolution (Å) | 79.62-2.18 | 38.80-1.94 |
| No. reflections | 34236 | 57144 |
| R_{work} / R_{free} | 17.70/24.80 | 19.80/23.10 |
| No. atoms | | |
| Protein | 8774 | 4502 |
| Water | 411 | 347 |

B-factors

| | | |
|---|---|---|
| Protein | 49.37 | 48.57 |
| Water | 30.70 | 56.30 |

R.m.s. deviations

| | | |
|---|---|---|
| Bond lengths (Å) | 0.010 | 0.010 |
| Bond angles (°) | 1.22 | c |
| PROASIS ID | 1txqc | |

*Values in parentheses are for highest-resolution shell.

### Structure of Fab 0182-IL-6 Complex

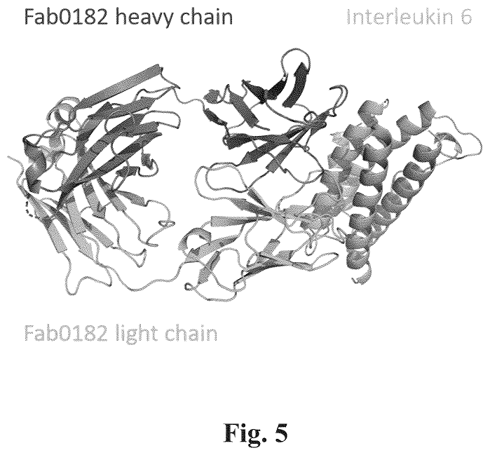

The crystal structure of the complex of Fab 0182 (representative of the VH6L-series of Fabs) in a complex with IL-6 was determined at a resolution of 2.18 Å (FIG. 5). The structure shows Fab 0182 bound to IL-6 by contributions of the CDR2 of the heavy and CDR1, CDR3 of the light chain. Further interactions with IL-6 are maintained by N-terminal residues Val3 and Gln4 of the light chain of Fab 0182. The interface contributed by IL-6 is formed by residues of helix A and helix C.

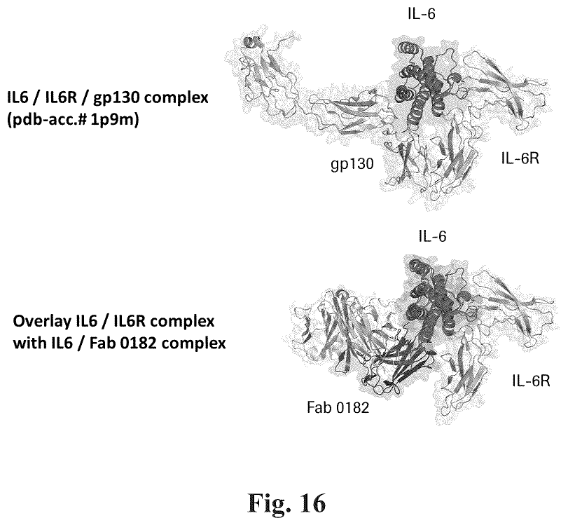

FIG. 16 illustrates the mode of binding of Fab 0182 to IL6. For the purpose of illustration, we generated an overlay of two structures: firstly, that of the complex structure of the Fab with IL6, and secondly, that of the complex structure of IL6R with IL6 (taken from the co-crystal structure of IL6, IL6R and gp130 with pdb accession code 1p9m, cf. Boulanger M J et al., Science 2003, 27; 300 (5628); 2101-4). Comparing this to the trimeric complex of IL6 with IL6R and gp130, it is apparent that the Fab binds to IL6 in a very similar fashion as gp130 does, i.e. it binds to the site 2 of IL6. This mode of binding is expected to allow the simultaneous binding of both the Fab and of IL6R to IL6, i.e. the interaction of IL6 with the IL6R should still be possible and such an IL6 antagonist a priori is expected to work by inhibiting the interaction of the IL6/IL6R complex with gp130 via binding to the site 2 of IL6.

### Structure of Fab 6HVL4.1-IL-6 Complex

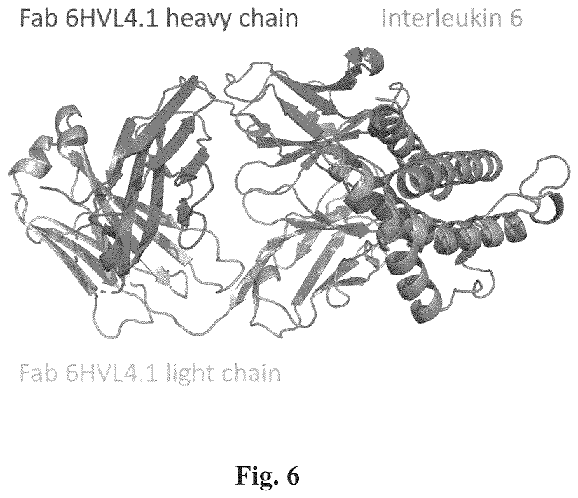

We determined the crystal structure of the complex of Fab 6HVL4. 1 in complex with IL-6 at a resolution of 1.94 Å (FIG. 6). The structure shows Fab 6HVL4.1 bound to IL-6 by main contributions of CDR1, CDR3 of heavy and CDR2, CDR3 of the light chain. Further interactions with IL-6 are maintained by the first three N-terminal residues of the heavy chain of Fab 6HVL4.1. The interface contributed by IL-6 is formed by residues of helix A and helix C.

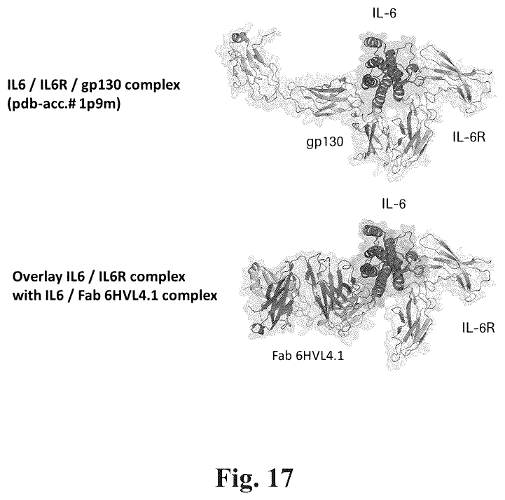

Similar to what was done for Fab 0182, we analyzed the mode of binding of Fab 6HVL4.1 to IL6 using an overlay of structures and by analyzing the interacting residues as described above. FIG. 17 demonstrates that also 6HVL4. 1 binds to IL6 in a very similar fashion as gp130 does and from a structural point of view therefore has to be considered as a site 2 binder.

### Experimental Investigation of the Mode of Binding to IL6

The fact that a clone like 6HVL4.1 and derivatives of it are IL6 site 2 binders could be confirmed functionally by assays utilizing surface plasmon resonance.

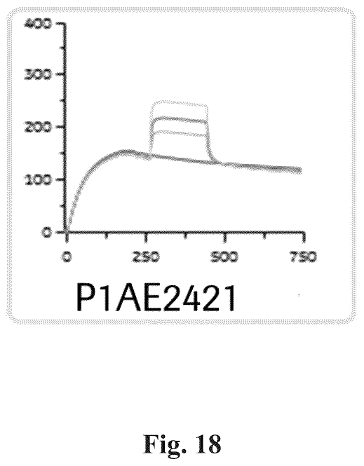

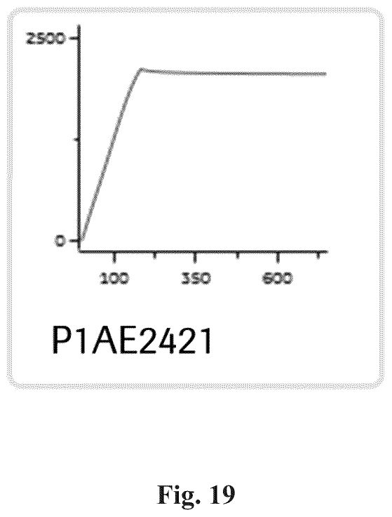

In one such assay, a Fab fragment representative of the 6HVL series of clones including 6HVL4.1 (antibody “PIAE2421”) was captured on the SPR chip's surface via an anti-Fab antibody, and IL6 and subsequently IL6R at three different concentrations (250, 500, 1000 nM) were flowed over the chip surface. Here, we expect a two-step, sequential signal increase if IL6 is capable of binding both to the Fab and IL6R. And indeed, for the Fab tested this is what was observed (FIG. 18); In the SPR signal trace, the addition of IL6 leads to a strong increase of the signal, which is further enhanced after addition of IL6R. This clearly demonstrates that a simultaneous binding of IL6R and the Fab to IL6 is possible. This finding is further corroborated by the fact that the covalent chimera of IL6 and IL6R (termed “hyper-IL6”) where the site 1 of IL6 is completely shielded and inaccessible still facilitates binding of the respective Fab molecule when coated onto an SPR chip and probed with the Fabs at a concentration of 26 nM (FIG. 19).

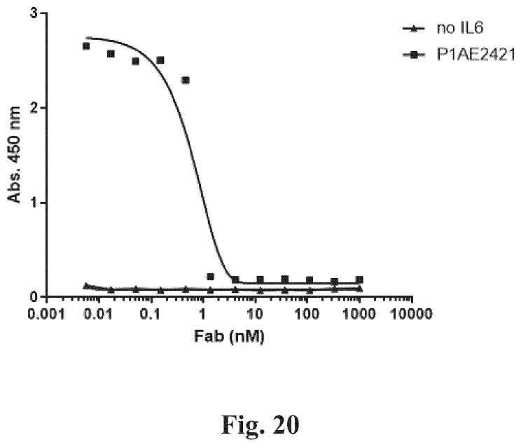

We obtain a surprising result, though, when utilizing an ELISA experiment to investigate whether the captured Fab fragment competes with the binding of IL6 to IL6R. The assay setup was as follows: first, a constant concentration of IL6 was preincubated with a titration series of the Fab fragment PIAE2421 that was also used in the SPR experiments and which was representative of 6HVL series of clones. This was then incubated on an ELISA plate that was directly coated with IL6R. After washing, IL6 that was bound by plate-bound IL6R was detected with a biotinylated anti-IL6 antibody utilizing horse-radish-peroxidase-labeled streptavidin (Strep-HRP). In this assay (FIG. 20), we observe a result that very much suggests a nearly complete inhibition of the IL6/IL6R interaction by the Fab.

Given that we know from the available crystal structure and the SPR experiments that the site 1 of IL6 is still accessible for binding, we have to interpret these results such that the IL6 antibodies described in this patent are capable not only of sterically blocking the binding of the IL6/IL6R complex to gp130, but to also allosterically strongly reducing the binding affinity of IL6 to IL6R, i.e. to functionally act as a an IL6 site 1 antagonist in addition.

Such a mode of action is expected to have optimal properties for the following reasons:
1. Due to being a IL6 site 2 binder, the IL6 antagonist is capable of blocking equally effectively the formation of a signaling complex by IL6 binding to membrane-bound IL6R and gp130 (cis-signaling) or of the preformed complex of IL6 and IL6R (trans-signaling). In contrast, an IL6 site 1 binder can not bind to the pre-formed complex of IL6 and IL6R but can only antagonize it if the complex dissociates.
2. Due to allosterically reducing the affinity of IL6 to IL6R, the IL6 antagonists described here are expected to display an increased potency compared to a site 2 binder that does not exhibit this effect, by disfavouring the formation of the first step of signaling, i.e. the formation of the IL6/IL6R complex. A site 2 binder not allosterically interfering with site 1 binding is expected to have a disadvantage particularly for cis-signaling, i.e. when having to block the second step of IL6/IL6R-gp130 complex formation on the surface of a cell, where the relative effective concentrations of IL6/IL6R and gp130 are expected to be very high.
3. IL6 site 1 binders, when used systemically as an antibody, are known to lead to a strong accumulation of the complex of IL6 with the antibody due to a strong increase in the half-life of the complex compared to IL6 alone. IL6 site 2 binders, in contrast, are expected to still allow the elimination of the IL6/antibody complex by binding to membrane-bound IL6R and subsequent internalization and degradation of the cell taking up the complex. In that regard, the IL6 antagonists described here are expected to combine the desirable properties both of a site 1 and a site 2 binder: while functionally being able to block the first step of the formation of the IL6/IL6R/gp130 signaling complex, they still allow degradation of the IL6/mAb complex via IL6R binding on cells.
4. In ophthalmic indications and utilized as a Fab molecule, the expected behavior of such binders may still be more beneficial: Similar to IL6 site 1 binders, the Fab/IL6 complex can leave the ophthalmic space relatively unhindered by IL6R binding and can be quickly eliminated systemically by renal filtration.

### Example 8

### Thermal Stability of Improved Anti-VEGF/Anti-IL6 Fab Fragments

Further sequence variants of improved anti-VEGF/anti-IL-6 antibodies were generated, comprising the amino acid sequences identified in Table 8.

TABLE 8
Amino acid sequences of indicated bispecific Fab fragments
(the numbers refer to the SEQ ID NOs as used herein)

| | VL | VH |
|---|---|---|
| 6HVL_5 | 39 | 40 |
| 6HVL_6 | 41 | 42 |
| VH6L_4 | 43 | 44 |
| VH6L_5 | 45 | 46 |

Thermal stability of the indicated bispecific antibodies was assessed as follows.

### Thermal Stability:

Samples of the bispecific antibody Fab fragments were prepared at a concentration of 1 mg/mL in 20 mM Histidine/Histidine chloride, 140 mM NaCl, pH 6.0, and transferred to a 10 μL micro-cuvette array. Static light scattering data as well as fluorescence data upon excitation with a 266 nm laser are recorded using an UNcle instrument (Unchained Labs), while samples are heated at a rate of 0.1° C./min from 30° C. to 90° C. Samples were measured in triplicates.

The evaluation of the onset temperatures was done by the UNcle analysis software. The aggregation onset temperature is defined as the temperature at which the scattered light intensity starts to increase. The denaturation of the protein was monitored by the shift in the barycentric mean (BCM) of the fluorescence signal over the thermal. The melting temperature is defined as the inflection point of the BCM (nm) vs. temperature curve. 20

TABLE 9
Thermal stability

| | Tagg (° C.) | Tm |
|---|---|---|
| 6HVL_4 | 64.7 | 70.1 |
| 6HVL_5 | 64.9 | 71.3 |
| 6HVL_6 | 63.4 | 66.8 |
| VH6L_3 | 65.8 | 77.0 |
| VH6L_4 | 66.6 | 76.3 |
| VH6L_5 | 66.6 | 77.0 |

### Example 9

### Biophysical Properties of Improved Bispecific Anti-VEGF/Anti-IL6 Fab Fragments (Viscosity Assessment by Dynamic Light Scattering (DLS))

Antibody Fab fragments as described before were expressed in CHO cells by standard methods.

Viscosity was measured with the latex-bead DLS method as described before (He F et al.; Anal Biochem. 2010 Apr. 1; 399 (1); 141-3). Specifically, the following protocol was followed using the indicated materials.

### Viscosity Assessment:

### Instrumentation and Materials

Wyatt DLS plate reader with a Greiner Bio-One microplate
3000 Series Nanosphere™Size Standards (Thermofisher Cat.-No. 3300 Å)
Tween 20 (Roche, cat.no. 11332465001) and silicone oil e.g. (Alfa Aesar cat.no. A12728)
UV photometer for concentration determination (e.g. Nanodrop 8000).

### Sample Preparation

Antibody samples were re-buffered and diluted in 20 mM His/HCl, pH 5.5 (buffer) and 0.02% Tween 20 (final concentration). A bead concentration of 0.03% solids was added. At least three different concentrations were prepared, where possible the highest concentration was about 200 mg/mL. Two blank samples were required as antibody-free controls: one comprising the Nanosphere beads resuspended in water, and another one comprising the Nanosphere beads resuspended in buffer. Samples were transferred into the micro plate and each well was covered with silicone oil.

Measurements with Wyatt DLS Plate Reader

All samples and the blanks were analyzed at different temperatures, from 15 to 35° C. in 5° C. steps. The acquisition time was 30s and the number of acquisitions was 40 per sample and temperature.

### Data Analysis

The raw data Dapp (apparent radii) in nm was shown in an overview of the software template (Microsoft Dynamics 7.10 or higher). The viscosity was calculated with a formula (ηreal=Dapp*ηH2O/Dreal). Dreal is the measured bead size in the blank sample, which is equal to the bead size (300 nm). The calculated viscosity was shown in Excel curves. With a Mooney curve Fit (in Excel), it is possible to extrapolate the viscosity at a given concentration. Here, the maximum protein concentration where the viscosity exceeds 20 cP was calculated.

The maximal concentration of the indicated antibodies to achieve a viscosity of 20 cP at 20° C. is indicated below.

TABLE 10
Viscosity by DLS bead method. Shown is the max.
feasible concentration of the indicated antibody
to reach 20 cP at 20° C.

| | Concentration [mg/ml] |
|---|---|
| 6HVL_4 | 195.4 |
| 6HVL_5 | 192.4 |
| VH6L_3 | 220.0 |
| VH6L_4 | 226.8 |
| VH6L_5 | 240.4 |

Results indicate that antibodies of the invention may be formulated in high concentrations comprising a viscosity below the acceptable viscosity limit for syringeability. In consequence, the antibodies of the invention are highly suitable for ocular application as they allow for provision of a high molar dose in a limited injection volume, which when combined with high potency results in a high durability and consequently, a reduced dosing frequency, which is desirable to increase patient convenience and treatment compliance.

### Example 10

### Primary Cell Based Assay to Demonstrate IL6 Inhibition Mediated by VEGF/IL6-Bispecific Antibody 6HVL_4 (HRMEC)

To measure IL-6 signaling activity in HRMECs, an assay quantifying ICAM-1 surface expression in HRMECs was established. HRMECs were stimulated with a combination of human IL-6 and human IL-6R at equimolar concentrations (2 nM) for 72 hours. ICAM-1 surface expression was assessed by flow cytometry. To measure the inhibitory activity of 6HVL_4, IL-6/IL-6R mixtures were pre-incubated with increasing concentrations of antibody prior to application to the cells.

Cell culture: HRMECs (Catalog No. PEL-PB-CH-160-8511; PELOBiotech Gmbh; Bayern, Germany) were thawed and cultured in a 175 cm² flask in endothelial growth medium (EGM-MV) comprising endothelial basal medium (EBM) (Catalog No. CC-3156; Lonza; Basel, Switzerland) and 5% fetal bovine serum (FBS), hydrocortisone, human fibroblast growth factor B, VEGF, R3-IGF-1 (recombinant analog of insulin-like growth factor-I with the substitution of Arg for Glu at position 3), ascorbic acid, human epidermal growth factor, and GA-1000 (all included in EGM-2 MV Microvascular Endothelial SingleQuots™ Kit; Catalog No. CC-4147; Lonza), at the manufacturer's recommended concentrations. Twenty-four hours after plating, the medium was changed with fresh EGM-MV and the cells were grown for 3 more days prior to the assay. Assay conditions were optimized across different passage numbers and concentrations of IL-6/soluble IL-6R. The final assay was performed using HRMECs at passage 6 and an equimolar stimulus of IL-6/soluble IL-6R at a concentration of 2 nM.

Flow cytometry assay; HRMECs were detached from the flask by washing twice with phosphate-buffered saline (PBS) without Ca2+ and Mg2+ (Catalog No. 10010023; Life Technologies) and once with cell dissociation reagent Accutase (Catalog No. A1110501; Thermo Fisher Scientific; Waltham, MA). After washing, 5 mL of the cell dissociation reagent was added to the cells and the flask was incubated at 37° C. in a 5% CO2 incubator for 3 minutes. Detached cells were collected from the flask and placed in a 50-ml conical centrifuge tube. The tube was filled to 50 mL with EBM containing 2% FBS and centrifuged at 300 g for 6 minutes. The supernatant was discarded, and the pellet was resuspended in 5 mL starvation medium (EBM containing 2% FBS). The cell number was quantified using TC20 Automated Cell Counter (Bio-Rad; Hercules, CA) and adjusted to 300,000 cells/mL using starvation medium. Then, 100 μL of the cell suspension was added to each well of a Costar 96-well plate (Catalog No. 3596; Corning; Corning, NY) yielding 30,000 cells/well. Afterwards, the plate was incubated at 37° C. in a 5% CO₂ incubator for another 24 hours.

Recombinant human IL-6 (Catalog No. 206-IL/CF; R&D Systems; Minneapolis, MN) and recombinant human IL-6R (Catalog No. 227-SR-025; R&D Systems) were mixed in starvation medium at equal molar concentrations and incubated at room temperature for 1 hour to allow formation of IL-6-I/L-6R complex. Next, 50 L of a dilution series (3-fold, 7-point dilution) of 6HVL_4 was added to the cells and incubated at 37° C., 5% CO₂, for 1 hour. Finally, 50 μL of the IL-6-I/L-6R complex was added to the cells yielding final concentrations of 2 nM each for IL-6 and IL-6R and 200.009 nM for 6HVL_4. Unstimulated cells and cells stimulated with IL-6-I/L-6R complex without 6HVL_4 were also included to determine the background ICAM-1 surface expression and the 100% response level, respectively. Cells were incubated at 37° C. in a 5% CO₂ incubator for 72 hours.

For analysis of ICAM-1 surface expression, cells were washed twice with PBS (Ca²⁺, Mg²⁺; Life Technologies) and once with cell dissociation reagent Accutase (Catalog No. A1110501; Thermo Fisher Scientific). Cells were detached from the plate using 50 μL of the cell dissociation reagent (3 minutes, 37° C.) and transferred to a flow cytometry Falcon 96-well Storage plate (Catalog No. 353263; Corning). The original wells were washed once with 100 μL PBS containing 2% FBS and 2 mM EDTA, and the wash medium containing the remaining cells was added to the flow cytometry plate. Cells were pelleted by centrifugation at 300 g for 6 minutes, and the supernatant was discarded. Pellets were resuspended in 100 μL PBS, 2% FBS, 2 mM EDTA containing 10 μg/mL human IgG (Catalog No. 12511; MilliporeSigma; Burlington; MA) to block nonspecific binding sites and incubated at room temperature for 15 minutes. Following blocking, 0.5 μg of fluorescein-labeled anti-ICAM-1 antibody (Catalog No. BBA20; R&D Systems) was added to the cells and the reaction was incubated at 28° C. for 45 minutes. Following staining, cells were pelleted by centrifugation at 300 g for 6 minutes and the pellet was resuspended in 150 μL PBS containing 2% FBS and 2 mM EDTA. Fluorescein fluorescence was measured using an Attune N×T flow cytometer (Thermo Fisher Scientific).

Data analysis: For all 3 assays, each condition in every independent experiment was performed in quadruplicates. For each experiment, the background signal of unstimulated cells was subtracted from that of the experimental wells and the mean signal per condition was calculated. A 100% response level was calculated from cells stimulated with IL-6/IL-6R without 6HVL_4, and the inhibitory potential of 6HVL_4 was then expressed as the percentage inhibition of the 100% response. The percentage inhibition for each concentration of 6HVL_4 was measured in 3 independent experiments, and the mean and SEM were calculated. The mean of the 3 independent experiments was used to calculate the mean IC50 and SE using ExcelXLfit software version 5.5.0 (IDBS; Guildford, UK). Concentration-response curves were fitted by nonlinear regression analysis using a 5-parameter logistic model (A+((B−A)/(1+(((B−E)*((C/x)\hat{ }D))/(E−A))))).

TABLE 11
Calculated % Inhibition of IL-6-Induced Expression of
ICAM-1 on the Surface of HRMECs by 6HVL_4

| concentration | Inhibition [%] | SEM [%] |
|---|---|---|
| 20 | 97.60 | 1.48 |
| 6.667 | 97.77 | 1.46 |
| 2.222 | 80.01 | 17.29 |
| 0.741 | 6.76 | 5.77 |
| 0.247 | −0.63 | 1.79 |
| 0.082 | −1.25 | 2.86 |
| 0.027 | −0.42 | 3.20 |

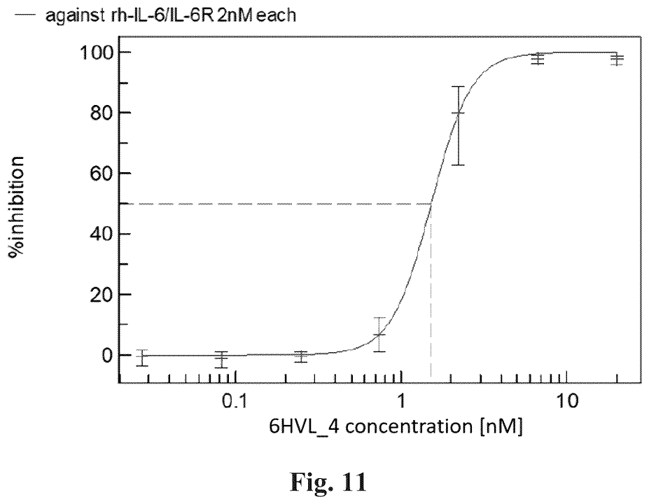

6HVL_4 led to a dose dependent inhibition of IL-6 signaling in HRMECs, with a 50% inhibitory concentration (IC50) of 1.52+/−0.04 nM (FIG. 11).

### Example 11

### Primary Cell Based Assay to Demonstrate VEGF Inhibition Mediated by VEGF/IL6-Bispecific Antibody 6HVL_4 (HUVEC)

### Assay:

HUVECs were obtained from Lonza (Catalog No. 00191027; Basel, Switzerland). Endothelial basal medium (EBM-2; Catalog No. CC-3156) and EGM-2 Endothelial SingleQuots Kit (Catalog No. CC4176), which together compose the endothelial growth medium (EGM-2) and assay medium (EBM-2 with 0.5% fetal bovine serum [FBS]), were also purchased from Lonza.

T175 cell culture flasks (Catalog No. 353112; Corning; Corning, NY) coated with attachment factor (AF) (Catalog No. S-006-100; Gibco, Thermo Fisher Scientific; Waltham, MA) were used for maintaining HUVECs. StemProAccutase (Catalog No. A11105-01; Gibco) was used to detach cells.

The cell viability/proliferation assay was performed in 96-well, fibronectin-coated plates (Catalog No. 354409; Corning) using alamarBlue (Catalog No. DAL1100; Invitrogen, Thermo Fisher Scientific).

Recombinant human VEGF-A was obtained from R&D (Catalog No. 293-VE; Minneapolis, MN) and dissolved in phosphate-buffered saline (PBS) without Ca²⁺ and Mg²⁺ (Catalog No. 14190-094; Gibco) at a stock concentration of 100 g/mL.

Alamarblue contains the cell permeable compound Resazurin. This compound changes its color due to the reducing environment within healthy cells. The pink color generated is a proportional marker of viable cells and can be used to detect proliferation by measuring absorbance at 570 nm. VEGF-A induces proliferation of HUVECs grown under cell starvation conditions. Therefore, VEGF-A-induced HUVEC proliferation can be inhibited by using a VEGF-A neutralizing antibody or Fab.

HUVECs were maintained in EGM-2 in T175 flasks coated with AF until passage 5. For the viability assay, HUVECs were detached using Accutase and diluted 1:1.66 in assay medium (EBM-2 0.5% FBS). Then, cells were centrifuged and resuspended in EBM-2 containing 0.5% FBS to a cell density of 100,000 cells/mL. Afterwards, 100 μL of the cell suspension was seeded on fibronectin-coated 96-well plates, thus yielding a cell density of 10,000 cells/well. Outer wells were not seeded with cells and were then filled with assay medium only. Cells were incubated overnight at 37° C. in a 5% CO₂ incubator.

The following day, a VEGF-A stock solution (100,000 ng/ml in PBS (Ca2+, Mg2+) was used to prepare a 10fold working solution (750 ng/ml) in assay medium (EBM 0.5% FBS).

6HVL_4 stock solution was also diluted in assay medium to prepare a 10fold working solution. This was used to prepare a 3-fold 8-point dilution series starting at 30,000 ng/mL and ending at 14 ng/ml.

Next, 12.5 μL of the 10 prediluted 6HVL_4 solution and 12.5 μL of the 10 VEGF-A solution (750 ng/mL) were sequentially added to the cells in quadruplicates in each plate. VEGF-A was used at constant final concentration (75 ng/ml), and 6HVL_4 was used in a dose-response format ranging from 3000 ng/ml to 1.4 ng/ml final concentration. Cells were incubated at 37° C., 5% CO₂, for 72 hours. For analysis, 12 μL of alamarBlue was added to each well and subsequently incubated in the cell culture incubator for 3 hours. Absorbance was measured at 570 nm, with a reference wavelength of 600 nm, using a FlexStation 3 plate reader from Molecular Devices.

### Data Analysis:

For each experiment, each condition was performed in quadruplicate. A total of 4 independent experiments were run. An independent experiment was considered as the processing of 2 separate plates on the same day. Therefore, 8 separate plates were used for analysis. The background signal of unstimulated cells was subtracted from that of the experimental wells, and the mean signal per condition was calculated. The 100% response level was calculated from cells stimulated with VEGF-A (75 ng/mL) without additional compound exposure, and the signal from 6HVL_4-exposed wells was expressed as the percentage inhibition of the 100% response.

IC50 values were calculated from the mean data for each antibody concentration using ExcelXLfit software version 5.5.0 (IDBS; Guildford, UK). Concentration-response curves were fitted by nonlinear regression analysis using a 4-parameter logistic model (A+((B−A)/(1+((C/x)\hat{ }D)))) calculated relative to basal and maximal inhibitory activity. Data are displayed as the average value from 4 independent experiments with the standard error of the mean (SEM).

TABLE 12
Calculated % Inhibition of the VEGF-A Induced
Proliferation of HUVECs by 6HVL_4

| concentration | Inhibition [%] | SEM [%] |
|---|---|---|
| 62.4 | 87.8 | 5.3 |
| 20.8 | 87.8 | 6.5 |
| 6.9 | 90.9 | 5.3 |
| 2.3 | 58.4 | 8.5 |
| 0.77 | 6.6 | 6.3 |
| 0.26 | 7.1 | 3.1 |
| 0.09 | 7.0 | 2.0 |

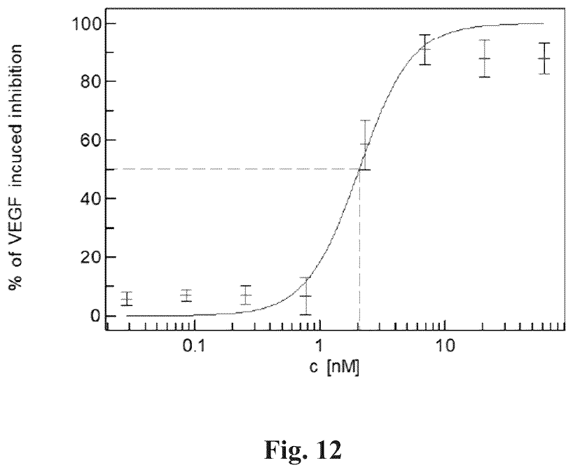

6HVL_4 reduced the VEGF-A-induced HUVEC proliferation, with a 50% inhibitory concentration (IC50) of 2.06+/−0.30 nM (FIG. 12).

### Example 12

### Restoration of Barrier Function in Presence of Both VEGF-A and IL6 and the VEGF/IL6 Bispecific Antibody 6HVL_4 Shows Biological Activity of the Molecule

In order to assess the dual biological activity of an antibody of the invention, i.e. the simultaneous blockade of both targeted cytokines-VEGF-A and IL6 (in complex with IL6R)—a trans-endothelial cell resistance (TER) assay was performed. In this assay, an electrically tight endothelial cell layer responds both to VEGF-A and IL6 addition by loss of barrier function. The restoration of barrier function in presence of both cytokines, VEGF and IL6, by the VEGF/IL6 bispecific antibody 6HVL_4 was assessed as follows:

### Assay:

Human Retinal Micro Vascular Endothelial Cells further named HRMVECs (PELOBiotech; Cat #PEL-PB-CH-160-8511) were maintained in complete MV-endothelial cell growth medium (MV-EGM-2 Lonza, Cat #CC-3202) in T175 flasks (Falcon Cat #353112), coated with Attachment Factor (Ginco, Cat #S-006-100) until passage 5. For the trans endothelial cell resistance assay cells were detached with StemPro®Accutase® (Gibco, Cat #A11105-01). Thereafter, cells were seeded in 100 μl MV-EGM-2 growth medium into the upper chamber of fibronectin-coated (Cat #354008, Corning) Transwell filters (24well Corning, Cat #3470) at a cell density of 120.000 cells/well. The lower chamber of the Transwell filter was filled with 600 μl of MV-EGM-2 medium. Cells were incubated for 3 day at 37° C. with 5% CO₂. After that the medium was changed to assay condition (MV-EGM-2 without VEGF containing 2% FBS) and the Transwell filters were transferred to the cellZcope system using 280 μl medium for the upper chambers and 810 μl for the lower compartments. Then the cells were incubated for 24 h at 37° C. with 5% CO₂ while the TER was measured by the cellZcope. The following day cells were treated with a final concentration of 10 ng/ml VEGF (R&D Systems, Cat #293-VE/CF), 50 ng/ml IL6 (R&D Systems, Cat #206-IL/CF) in combination with 100 ng/ml IL6R (R&D Systems, Cat #227-SR-025/CF) and 10 ng/ml VEGF or with an equal amount of assay medium (eightfold per condition) and the TER was measured until the next day. Thereafter 6HVL_4 or Aflibercept with a final concentration of 1 μg/ml respectively 2.3 μg/ml or Assay Medium were added to the cells, subsequently TER was measured for the next 24 h. Therefore each condition is present four times.

### Data Analysis:

Datasets generated for one well were normalized to the TER value obtained shortly before the addition of the cytokine mix. For each condition the mean signal and the standard deviation were calculated from the normalized data.

### Results:

Results are shown in FIGS. 12 (6HVL_4) and 13 (Aflibercept).

The barrier function of HRMVECs is reduced with the cytokines VEGF alone as well as in combination with IL6/IL6R. With antibody 6HVL_4 the destroyed barrier is recovered to 100% after 24h.

### Example 13

### Identification of IL6 Paratope Region

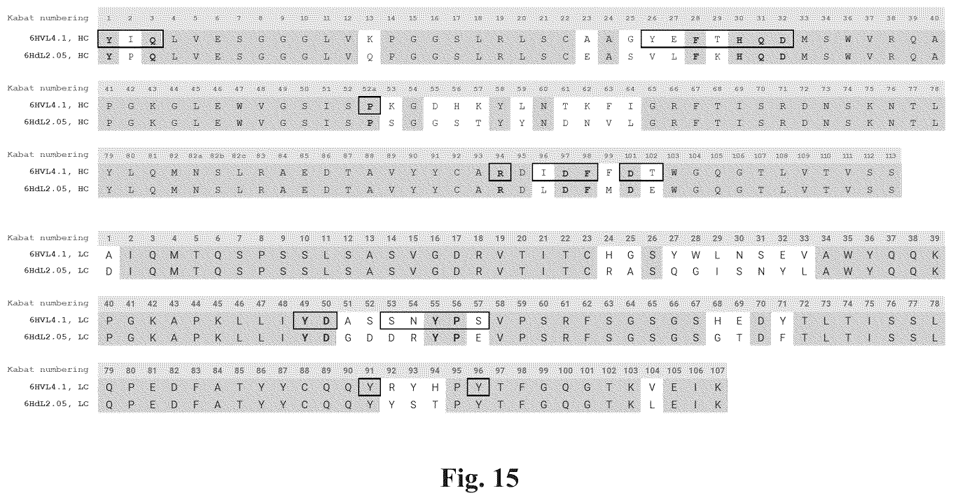

Amino acid residues in contact with IL6 were identified from the crystal structure of the complex between the 6HVL4.1 and IL6. An illustration of the position of paratope amino acid residues within the VH and VL domains is depicted in FIG. 15. For this purpose, residues likely to interact with IL6 in the Fab/IL6 complex were identified using the “byres” function of PyMOL and a cut-off distance of 5 Angstrom. Here, we restricted the analysis to residues 48-215 of IL6 (as defined in Uniprot ID P05231), which are the residues found to be usually resolved in structures of IL6 alone (cf. pdb accession #lalu and 1IL6). In FIG. 15, also an alignment is shown between 6HVL4.1 and a monospecific anti-IL6 antibody, 6HdL2.05, which is based on the antibody of the invention, wherein the VEGF-paratope is replaced by a non-binding region. 6HdL2.05 has a VH domain of SEQ ID NO: 48 and a VL domain of SEQ ID NO: 47. When expressed and purified as described in Example 2 and subjected to an SPR assay with human IL6 or cynomolgus monkey IL6 carried out in analogy to Example 3, the antibody exhibited SPR sensorgrams as depicted in

FIG. 20. After fitting of the experimental data, 6HdL2.05 displayed an affinity comparable to the highest affinities obtained for the corresponding 6HVL series of VEGF/IL6 bispecific antibodies (cf. Table 3 and Table 4), with a fitted KD of 22 pM with human IL6 and a fitted KD of 1.3 nM with cynomolgus monkey IL6.

The amino acid residues identified to contribute to antigen binding are identified in Table 13 (for the variable heavy chain domain amino acid residues) and Table 14 (for the variable light chain domain amino acid residues). Amino acid positions are numbered according to the Kabat numbering system (the same numbering is used in FIG. 1+5). Amino acids positions involved in antigen binding are identified by their Kabat position in the VH or VL domain.

TABLE 13
Variable domain amino acid residues involved in
IL6 binding as identified by crystal structure
analysis, amino acid residues at same Kabat
position are shown for 6HdL2.05

| domain | 6HVL4.1 | 6HdL2.05 |
|---|---|---|
| VH | Y1, I2, Q3, | Y1, P2, Q3, |
| | Y26, E27, F28, T29, | V26, L27, F28, K29, |
| | H30, Q31, D32, | H30, Q31, D32, |
| | P52a, | P52a, |
| | R94, I96, D97, | R94, L96, D97, |
| | F98, D101, T102 | F98, D101, E102 |
| VL | Y49, D50, S53, N54, Y55, P56, | Y49, D50, D53, R54, |
| | S57, | Y55, P56, E57, |
| | Y91, Y96 | Y91, Y96 |

1. An antibody that binds to human VEGF-A and to human IL6 comprising a VH domain comprising (a) CDR-H1 comprising the amino acid sequence of SEQ ID NO: 18, (b) CDR-H₂ comprising the amino acid sequence of SEQ ID NO: 19, and (c) CDR-H₃ comprising the amino acid sequence of SEQ ID NO: 20, and a VL domain comprising (d) CDR-LI comprising the amino acid sequence of SEQ ID NO:15, (e) CDR-L2 comprising the amino acid sequence of SEQ ID NO: 16, and (f) CDR-L3 comprising the amino acid sequence of SEQ ID NO:17, comprising a variable heavy chain domain comprising an amino acid sequence of SEQ ID NO:22 with up to 5 amino acid substitutions; and a variable light chain domain comprising an amino acid sequence of SEQ ID NO: 21 with up to 5 amino acid substitutions.

2. The antibody according to claim 1, wherein the up to 5 amino acid substitutions occur in the FR region of the respective variable domain.

3. The antibody according to claim 1 or 2, comprising a VH sequence of SEQ ID NO: 22 and a VL sequence of SEQ ID NO:21.

4. The antibody of one of the preceding claims, comprising a heavy chain amino acid sequence of SEQ ID NO:24 and a light chain amino acid sequence of SEQ ID NO: 23.

5. An antibody that binds to human VEGF-A and to human IL6, comprising a VH sequence of SEQ ID NO:22 and a VL sequence of SEQ ID NO:21.

6. An antibody that binds to human IL6 that binds to the same epitope on IL6 as an antibody with a VL domain of SEQ ID NO: 35 and a VH domain of SEQ ID NO: 36.

7. The antibody of any one of the preceding claims, wherein the antibody is a Fab fragment.

8. The antibody of any one of the preceding claims, wherein the antibody is a bispecific antibody fragment.

9. An isolated nucleic acid encoding the antibody of any of claims 1 to 8.

10. A host cell comprising the nucleic acid of claim 9.

11. A method of producing an antibody that binds to human VEGF-A and to human IL6 comprising culturing the host cell of claim 10 so that the antibody is produced.

12. The method of claim 11, wherein the host cell is a CHO cell.

13. A pharmaceutical formulation comprising the antibody of any one of claims 1 to 8 and a pharmaceutically acceptable carrier.

14. A port delivery device comprising the antibody of any one of claims 1 to 8.

15. The antibody of any one of claims 1 to 8 for use as a medicament.

16. A port delivery device comprising the antibody of any of claims 1 to 8 or the pharmaceutical formulation of claim 13.


SEQUENCE LISTING (48 sequences)

SEQ ID NO: 1 - protein, 107 aa, synthetic construct
  REGION 1..107: note = VL VH6L-BM
NIQKTQSPSSLSASVGDRVTITCRALWHISSYLAWYQQKPGKAPKLLIYAASHLHYGVPSRFSGSGSGTDFTLTISSLQPEDFATYYCQQYSGLPYTFGQGTKVEIK

SEQ ID NO: 2 - protein, 116 aa, synthetic construct
  REGION 1..116: note = VH VH6L-BM
YYQLVESGGGLVQPGGSLRLSCAADGFLFSGYAMSWIRQAPGKGLEWIGQISGSGGSTVYNYNVLGRFTISRDNSKNTLYLQMNSLRAEDTAVYYCARDTGYFDHWGQGTLVTVSS

SEQ ID NO: 3 - protein, 106 aa, synthetic construct
  REGION 1..106: note = VL 6HVL_1
DQMTQSPSSLSASVGDRVTITCNAWWNISNYLAWYQQKPGKAPKLLIFDAKYRAPEVPSRFSGSGSGSQFTLTISSLQPEDFATYYCQQYSGWPFTFGQGTKVEIK

SEQ ID NO: 4 - protein, 106 aa, synthetic construct; identical to SEQ ID NO: 3
  REGION 1..106: note = VH 6HVL_1

SEQ ID NO: 5 - protein, 106 aa, synthetic construct
  REGION 1..106: note = VL 6HVL_2
DQMTQSPSSLSASVGDRVTITCKALWEVRDYLAWYQQKPGKAPKLLIFDGEYRAPEVPSRFSGSGSGTEFTLTISSLQPEDFATYYCQHYAGWPYTFGQGTKLEIK

SEQ ID NO: 6 - protein, 115 aa, synthetic construct
  REGION 1..115: note = VH 6HVL_2
GQLVESGGGLVQPGGSLRLSCAASGFYWGPGQMSWVRQAPGKGLEWVGSIDTKGDDEWYDPKFDGRFTISRDNAKNSLYLQMNSLRAEDTAVYYCARDIGFFDTWGQGTLVTVSS

SEQ ID NO: 7 - protein, 106 aa, synthetic construct
  REGION 1..106: note = VL 6HVL_3
IQMTQSPSSLSASVGDRVTITCRALWEIRDYLAWYQQKPGKAPKLLIFDGEYRAPEVPSRFSGSGSGTDFTLTISSLQPEDFATYYCQHYAGWPYTFGQGTKLEIK

SEQ ID NO: 8 - protein, 115 aa, synthetic construct
  REGION 1..115: note = VH 6HVL_3
GQLVESGGGLVQPGGSLRLSCAASGFYWGPGQMSWVRQAPGKGLEWVGSIDTKGRDEWYDPKFDGRFTISRDNSKNTLYLQMNSLRAEDTAVYYCARDIGFFDTWGRGTLVTVSS

SEQ ID NO: 9 - protein, 107 aa, synthetic construct
  REGION 1..107: note = VL VH6L_1
AIQMTQSPSSLSASVGDRVTITCHGSYWLSSEVAWYQQKPGKAPKLLIYDGDDVVPEVPSRFSGSGSHEDYTLTISSLQPEDFATYYCQQYRYHPYTFGHGTKVEIK

SEQ ID NO: 10 - protein, 115 aa, synthetic construct
  REGION 1..115: note = VH VH6L_1
YIQLVESGGGLVKPGGSLRLSCAAGYEFTHQDMSWVRQAPGKGLEWVGSISPKGDHKYLNTKFIGRFTISRDNSKNTLYLQMNSLRAEDTAVYYCARDIGFFDTWGQGTLVTVSS

SEQ ID NO: 11 - protein, 107 aa, synthetic construct
  REGION 1..107: note = VL VH6L_2
AIQMTQSPSSLSASVGDRVTITCHGSYWLSSEVAWYQQKPGKAPKLLIYDASSNYPSVPSRFSGSGSHEDYTLTISSLQPEDFATYYCQQYRYHPYTFGHGTKVEIK

SEQ ID NO: 12 - protein, 115 aa, synthetic construct
  REGION 1..115: note = VH VH6L_2
YIQLVESGGGLVKPGGSLRLSCAAGYEFTHQDMSWVRQAPGKGLEWVGSISPKGDHKYLNTKFIGRFTISRDNSKNTLYLQMNSLRAEDTAVYYCARDIDFFDTWGQGTLVTVSS

SEQ ID NO: 13 - protein, 107 aa, synthetic construct
  REGION 1..107: note = VL VH6L_3
AIQMTQSPSSLSASVGDRVTITCHGSYWLNSEVAWYQQKPGKAPKLLIYDGDTVYPEVPSRFSGSGSHEDYTLTISSLQPEDFATYYCQQYRYHPYTFGQGTKVEIK

SEQ ID NO: 14 - protein, 115 aa, synthetic construct
  REGION 1..115: note = VH VH6L_3
YPQLVESGGGLVKPGGSLRLSCAASVLFKHQDMSWVRQAPGKGLEWVGSISPKGDHKYLNTKFIGRFTISRDNSKNTLYLQMNSLRAEDTAVYYCARDIDFFDEWGQGTLVTVSS

SEQ ID NO: 15 - protein, 11 aa, synthetic construct
  REGION 1..11: note = LCDR-1 6HVL_4
QASYWLNSELA

SEQ ID NO: 16 - protein, 7 aa, synthetic construct
  REGION 1..7: note = LCDR-2 6HVL_4
DGDDRYP

SEQ ID NO: 17 - protein, 9 aa, synthetic construct
  REGION 1..9: note = LCDR-3 6HVL_4
QQYRYTPYT

SEQ ID NO: 18 - protein, 4 aa, synthetic construct
  REGION 1..4: note = HCDR-1 6HVL_4
QDMS

SEQ ID NO: 19 - protein, 17 aa, synthetic construct
  REGION 1..17: note = HCDR-2 6HVL_4
SIDPRGDHKYYNTKFID

SEQ ID NO: 20 - protein, 7 aa, synthetic construct
  REGION 1..7: note = HCDR-3 6HVL_4
DLDFMDE

SEQ ID NO: 21 - protein, 107 aa, synthetic construct
  REGION 1..107: note = VL 6HVL_4
AIYMHQEPSSLSASVGDRVTITCQASYWLNSELAWYQQKPGKAPKLLIYDGDDRYPEVPSRFSGSGSHEDYTLTISSLQPEDFATYYCQQYRYTPYTFGQGTKLEIK

SEQ ID NO: 22 - protein, 115 aa, synthetic construct
  REGION 1..115: note = VH 6HVL_4
YPQLVESGGGLVQPGGSLRLSCEASVLFKHQDMSWVRQAPGKGLEWVGSIDPRGDHKYYNTKFIDRFTISRDNSKNTLYLQMNSLRAEDTAVYYCARDLDFMDEWGQGTLVTVSS

SEQ ID NO: 23 - protein, 214 aa, synthetic construct
  REGION 1..214: note = LC 6HVL_4
AIYMHQEPSSLSASVGDRVTITCQASYWLNSELAWYQQKPGKAPKLLIYDGDDRYPEVPSRFSGSGSHEDYTLTISSLQPEDFATYYCQQYRYTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 223 aa, synthetic construct
  REGION 1..223: note = HC 6HVL_4
YPQLVESGGGLVQPGGSLRLSCEASVLFKHQDMSWVRQAPGKGLEWVGSIDPRGDHKYYNTKFIDRFTISRDNSKNTLYLQMNSLRAEDTAVYYCARDLDFMDEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 25 - protein, 107 aa, synthetic construct
  REGION 1..107: note = VL 6HVL_4 YHE
AIQMTQSPSSLSASVGDRVTITCQASYWLNSELAWYQQKPGKAPKLLIYDGDDRYPEVPSRFSGSGSHEDYTLTISSLQPEDFATYYCQQYRYTPYTFGQGTKLEIK

SEQ ID NO: 26 - protein, 115 aa, synthetic construct; identical to SEQ ID NO: 22
  REGION 1..115: note = VH 6HVL_4 YHE

SEQ ID NO: 27 - protein, 147 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 28 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 29 - protein, 107 aa, Homo sapiens [sequence omitted]

SEQ ID NO: 30 - protein, 108 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 31 - protein, 108 aa, synthetic construct
  REGION 1..108: note = Fab 0182 VL
TPVQMTQSPSSLSASVGDRVTITCHGFWDIRDYLAWYQQKPGKAPKLLIFDAKYRAPEVPSRFSGSGSGTEFTLTISSLQPEDFATYYCQQYSGWPFTFGQGTKVEIK

SEQ ID NO: 32 - protein, 115 aa, synthetic construct
  REGION 1..115: note = Fab 0182 VH
GQLVESGGGLVKPGGSLRLSCKASGFYWGPGAMSWVRQAPGKGLEWVGSIDLKGEDAWFDHEFSGRFTISRDNSKDTLYLQMNSLRAEDTAVYYCARDIGFFDTWGQGTLVTVSS

SEQ ID NO: 33 - protein, 215 aa, synthetic construct
  REGION 1..215: note = Fab 0182 LC
TPVQMTQSPSSLSASVGDRVTITCHGFWDIRDYLAWYQQKPGKAPKLLIFDAKYRAPEVPSRFSGSGSGTEFTLTISSLQPEDFATYYCQQYSGWPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 223 aa, synthetic construct
  REGION 1..223: note = Fab 0182 HC
GQLVESGGGLVKPGGSLRLSCKASGFYWGPGAMSWVRQAPGKGLEWVGSIDLKGEDAWFDHEFSGRFTISRDNSKDTLYLQMNSLRAEDTAVYYCARDIGFFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 35 - protein, 107 aa, synthetic construct
  REGION 1..107: note = 6HVL4.1 VL
AIQMTQSPSSLSASVGDRVTITCHGSYWLNSEVAWYQQKPGKAPKLLIYDASSNYPSVPSRFSGSGSHEDYTLTISSLQPEDFATYYCQQYRYHPYTFGQGTKVEIK

SEQ ID NO: 36 - protein, 115 aa, synthetic construct; identical to SEQ ID NO: 12
  REGION 1..115: note = 6HVL4.1 VH

SEQ ID NO: 37 - protein, 251 aa, synthetic construct
  REGION 1..251: note = 6HVL4.1 LC
MTMITPSFGAFFLEIFNMKQSTIALALLPLLFTPVSKAAIQMTQSPSSLSASVGDRVTITCHGSYWLNSEVAWYQQKPGKAPKLLIYDASSNYPSVPSRFSGSGSHEDYTLTISSLQPEDFATYYCQQYRYHPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 38 - protein, 217 aa, synthetic construct
  REGION 1..217: note = 6HVL4.1 HC
YIQLVESGGGLVKPGGSLRLSCAAGYEFTHQDMSWVRQAPGKGLEWVGSISPKGDHKYLNTKFIGRFTISRDNSKNTLYLQMNSLRAEDTAVYYCARDIDFFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 39 - protein, 107 aa, synthetic construct
  REGION 1..107: note = VL 6HVL_5
AIYMHQEPSSLSASVGDRVTITCQASYWLNSELAWYQQKPGKAPKLLIYDGDTNYPEVPSRFSGSGSHEDYTLTISSLQPEDFATYYCQQYRYTPYTFGQGTKLEIK

SEQ ID NO: 40 - protein, 115 aa, synthetic construct; identical to SEQ ID NO: 22
  REGION 1..115: note = VH 6HVL_5

SEQ ID NO: 41 - protein, 107 aa, synthetic construct
  REGION 1..107: note = VL 6HVL_6
AIYMHQEPSSLSASVGDRVTITCQASYWLNSELAWYQQKPGKAPKLLIYDGDDTYPEVPSRFSGSGSHEDYTLTISSLQPEDFATYYCQQYRYTPYTFGQGTKLEIK

SEQ ID NO: 42 - protein, 115 aa, synthetic construct
  REGION 1..115: note = VH 6HVL_6
YPQLVESGGGLVQPGGSLRLSCEASVLFKHQDMSWVRQAPGKGLEWVGSISPRGDHKYYNTKFIDRFTISRDNSKNTLYLQMNSLRAEDTAVYYCARDLDFMDEWGQGTLVTVSS

SEQ ID NO: 43 - protein, 106 aa, synthetic construct
  REGION 1..106: note = VL VH6L_4
IQMTQSPSSLSASVGDRVTITCRALWEIRDYLAWYQQKPGKAPKLLIFDGEYRAPEVPSRFSGSGSGTEFTLTISSLQPEDFATYYCQHYAGWPYTFGQGTKLEIK

SEQ ID NO: 44 - protein, 115 aa, synthetic construct
  REGION 1..115: note = VH VH6L_4
GQLVESGGGLVQPGGSLRLSCAASGFYWGPGQMSWVRQAPGKGLEWVGSIDTKGDDEWYDPKFDGRFTISRDNSKNTLYLQMNSLRAEDTAVYYCARDIGFFDTWGRGTLVTVSS

SEQ ID NO: 45 - protein, 107 aa, synthetic construct
  REGION 1..107: note = VLVH6L_5
AIQMTQSPSSLSASVGDRVTITCRALWEIRDYLAWYQQKPGKAPKLLIFDGEYRAPEVPSRFSGSGSGTDFTLTISSLQPEDFATYYCQHYAGWPYTFGQGTKLEIK

SEQ ID NO: 46 - protein, 115 aa, synthetic construct; identical to SEQ ID NO: 8
  REGION 1..115: note = VH VH6L_5

SEQ ID NO: 47 - protein, 214 aa, synthetic construct
  REGION 1..214: note = LC 6HDL2.05
DIQMTQSPSSLSASVGDRVTITCRASQGISNYLAWYQQKPGKAPKLLIYDGDDRYPEVPSRFSGSGSGTDFTLTISSLQPEDFATYYCQQYYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 240 aa, synthetic construct
  REGION 1..240: note = HC 6HDL2.05
YPQLVESGGGLVQPGGSLRLSCEASVLFKHQDMSWVRQAPGKGLEWVGSISPSGGSTYYNDNVLGRFTISRDNSKNTLYLQMNSLRAEDTAVYYCARDLDFMDEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPGSGMASMTGGQQMG